(19) United States
(12) Patent Application Publication
(10) Pub. No.: US 2024/0110234 A1
(43) Pub. Date: Apr. 4, 2024

(54) Amplification Compositions and Methods

(71) Applicant: Illumina, Inc., San Diego, CA (US)

(72) Inventors: Justin Robbins, San Diego, CA (US); Marie Hu, San Diego, CA (US)

(73) Assignee: Illumina, Inc., San Diego, CA (US)

(21) Appl. No.: 18/475,939

(22) Filed: Sep. 27, 2023

Related U.S. Application Data
(60) Provisional application No. 63/411,949, filed on Sep. 30, 2022.

(51) Int. Cl. C12Q 1/6844; C12Q 1/6874

(52) U.S. Cl. CPC C12Q 1/6844; C12Q 1/6874

(57) ABSTRACT

This disclosure relates to novel amplification compositions and methods, in particular for use in sequencing.

### CROSS-REFERENCE TO RELATED APPLICATION

This application claims the benefit of U.S. Provisional Patent Application No. 63/411,949, filed Sep. 30, 2022, and entitled “Amplification Compositions and Methods,” the disclosure of which is hereby incorporated by reference in its entirety.

### FIELD

This disclosure relates to novel amplification compositions and methods, in particular for use in sequencing.

### SEQUENCE LISTING

The instant application contains a Sequence Listing which has been submitted electronically in xml format and is hereby incorporated by reference in its entirety. Said xml copy was created on Sep. 22, 2023, is named 85491_08300_US.xml, and is 16.5 kilobytes in size.

### BACKGROUND

The detection of analytes such as nucleic acid sequences that are present in a biological sample has been used as a method for identifying and classifying microorganisms, diagnosing infectious diseases, detecting and characterising genetic abnormalities, identifying genetic changes associated with cancer, studying genetic susceptibility to disease, and measuring response to various types of treatment. A common technique for detecting analytes such as nucleic acid sequences in a biological sample is nucleic acid sequencing.

Advances in the study of biological molecules have been led, in part, by improvement in technologies used to characterise the molecules or their biological reactions. In particular, the study of the nucleic acids DNA and RNA has benefited from developing technologies used for sequence analysis.

Methods of nucleic acid amplification which allow amplification products to be immobilised on a solid support in order to form arrays comprised of clusters or “colonies” formed from a plurality of identical immobilised polynucleotide strands and a plurality of identical immobilised complementary strands are known. The nucleic acid molecules present in DNA colonies on the clustered arrays prepared according to these methods can provide templates for sequencing reactions.

One method for sequencing a polynucleotide template involves performing multiple extension reactions using a DNA polymerase to successively incorporate labelled nucleotides to a template strand. In such a “sequencing by synthesis” reaction a new nucleotide strand base-paired to the template strand is built up in the 5′ to 3′ direction by successive incorporation of individual nucleotides complementary to the template strand.

There remains a need to develop new amplification compositions and methods that increase throughput and accuracy of sequencing runs. The present disclosure addresses this need.

### SUMMARY

According to an aspect, there is provided an amplification composition (also referred to herein as the composition) comprising an inorganic polyphosphate and a polyphosphate kinase.

Preferably, the amplification composition may comprise at least one selected from the group consisting of: a recombinase, a single-stranded nucleotide binding protein, a polymerase and NTPs.

Preferably, the amplification composition comprises a recombinase.

Preferably, the amplification composition comprises a single-stranded nucleotide binding protein.

Preferably, the amplification composition comprises a polymerase.

Preferably, the amplification composition comprises NTPs.

Preferably, the amplification composition comprises a recombinase, a single-stranded nucleotide binding protein, a polymerase and NTPs.

Preferably, the amplification composition comprises the inorganic polyphosphate at a concentration of about 0.01 μM to about 1000 μM, about 0.1 μM to about 100 μM, about 0.5 μM to about 50 μM, about 1 μM to about 20 μM, or about 2 μM to about 10 μM.

Preferably, the amplification composition comprises the polyphosphate kinase at a concentration of about 0.01 μM to about 1000 μM, about 0.1 μM to about 100 μM, about 0.5 μM to about 50 μM, about 1 μM to about 20 μM, or about 2 μM to about 10 μM.

Preferably, the inorganic polyphosphate comprises a first inorganic polyphosphate with less than 50 phosphate residues.

Preferably, the inorganic polyphosphate comprises a second inorganic polyphosphate with more than 100 phosphate residues.

Preferably, a ratio of the first inorganic polyphosphate to the second inorganic polyphosphate is about 90:10 to about 10:90, about 80:20 to about 20:80, about 70:30 to about 30:70, about 60:40 to about 40:60, or about 50:50.

Preferably, the polyphosphate kinase is a thermophilic polyphosphate kinase.

Preferably, the polyphosphate kinase has an optimum working temperature of about 50° C. to about 75° C., about 55° C. to about 70° C., or about 60° C. to about 65° C.

Preferably, the polyphosphate kinase is selected from the group consisting of: a polyphosphate kinase of the PPK1 family, a polyphosphate kinase of the PPK2 family, and a polyphosphate kinase of the PPK3 family.

Preferably, the polyphosphate kinase comprises an amino acid sequence as defined in SEQ ID NO: 1 to 3, or a functional variant or functional fragment thereof.

Preferably, the composition does not comprise PEG.

Preferably, the amplification composition comprises a buffer.

Preferably, the amplification composition is buffered to a pH of about 6.0 to about 9.0, preferably about 6.5 to about 8.8, more preferably about 7.5 to about 8.7, even more preferably about 8.3 to about 8.6.

Preferably, the composition is a clustering composition or a sequencing-by-synthesis amplification composition or a resynthesis composition.

According to a further aspect, there is provided a kit comprising an inorganic polyphosphate and a polyphosphate kinase.

Preferably, the kit comprises an amplification composition as described herein.

Preferably, the kit may comprise at least one selected from the group consisting of: a recombinase, a single-stranded nucleotide binding protein, a polymerase and NTPs.

Preferably, the kit comprises a recombinase.

Preferably, the kit comprises a single-stranded nucleotide binding protein.

Preferably, the kit comprises a polymerase.

Preferably, the kit comprises NTPs.

Preferably, the kit comprises a recombinase, a single-stranded nucleotide binding protein, a polymerase and NTPs.

Preferably, the kit further comprises a metal cofactor composition, preferably wherein the metal cofactor composition comprises magnesium ions.

According to a further aspect, there is provided a use of an amplification composition as described herein, or a kit as described herein, in amplifying a nucleic acid template, or in sequencing a nucleic acid sequence.

According to a further aspect, there is provided a method of amplifying a nucleic acid template, wherein the method comprises recycling ADP to ATP using inorganic polyphosphate and a polyphosphate kinase.

Preferably, the method comprises adding an amplification composition as described herein.

Preferably, the method comprises adding a first polyphosphate composition and a second polyphosphate composition,
wherein in the first polyphosphate composition, an amount of the first inorganic polyphosphate is higher relative to an amount of the second inorganic polyphosphate, and
wherein in the second polyphosphate composition, an amount of the second inorganic polyphosphate is higher relative to an amount of the first inorganic polyphosphate.

Preferably, the first polyphosphate composition is added before the second polyphosphate composition.

Preferably, amplification is conducted by exclusion amplification.

According to a further aspect, there is provided a method of sequencing a nucleic acid sequence, wherein the method comprises:
amplifying a nucleic acid template using a method as described herein; and
sequencing the amplified nucleic acid template.

Preferably, the step of sequencing the amplified nucleic acid template comprises conducting a first sequencing read and a second sequencing read.

Preferably, the step of sequencing the amplified nucleic acid template is conducted using a sequencing-by-synthesis technique or a sequencing-by-ligation technique.

Preferably, the method is conducted isothermally.

Preferably, the method is conducted at a temperature of about 50° C. to about 75° C., about 55° C. to about 70° C., or about 60° C. to about 65° C.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1A shows a schematic representation of an example method of sequencing a nucleic acid according to examples of the present disclosure. FIG. 1B shows a typical inorganic polyphosphate (PolyPₙ) structure, where n is the number of phosphate residues. FIG. 1C shows a reaction scheme for the recycling of ADP using PolyPₙ catalysed by PPK1, to form ATP and an inorganic polyphosphate that has been reduced in the number of phosphate residues by 1 (i.e. to a number of phosphate residues of n−1).

FIG. 2 shows how PolyP can be enzymatically converted to ATP in the presence of ADP and the enzyme polyphosphate kinase 1 (PPK1), and how PolyP can be enzymatically converted to ATP in the presence of ADP and the enzyme polyphosphate kinase 1 (PPK2). Inorganic polyphosphate (PolyP) is a varying length high energy phosphoanhydride molecule that can be a donor for ATP generation. ATP is an essential small molecule within the amplification/clustering process. ATP supports the key functions of the recombinase including filament formation, homology, searching and invasion. ADP is a by-product of the recombinase enzymatic reactions, and a build-up of this small molecule can be inhibitory to the amplification/clustering reaction. ADP removal facilitated by PPK1/PPK2 enzymes removes this inhibitory molecule by recycling to generate ATP.

FIGS. 3A and 3B show expression and purification of recombinant Thermus thermophilus (Tth) PPK1 and Meiothermus ruber (Mm) PPK2. FIG. 3A—SDS-PAGE analysis of the Tth PPK1 and Mm PPK2 expression conditions. Lanes: (M) Marker with sizes denoted; (1) Uninduced—PPK1; (2) Induced 37° C. soluble—PPK1; (3) Pellet 37° C.—PPK1; (4) Induced 18° C. soluble—PPK1; (5) Pellet 18° C.—PPK1; (6) Uninduced—PPK2; (7) Induced 37° C. soluble—PPK2; (8) Pellet 37° C.—PPK2; (9) Induced 18° C. soluble—PPK2; (10) Pellet 18° C.—PPK2. FIG. 3B—SDS-PAGE analysis of the IMAC purification of Mm PPK2. Lanes: (M) Marker with sizes denoted; (1) Clarified soluble applied to the column; (2) FT; (3) Wash; (4) eluted fraction 1; (5) elution fraction 2; (6) elution fraction 3; (7) elution fraction 4.

FIG. 4 shows how PPi can be enzymatically converted to ATP in the presence of ADP and the enzyme polyphosphate kinase 1 (PPK1), and how PPi can be enzymatically converted to ATP in the presence of ADP and the enzyme polyphosphate kinase 2 (PPK2).

FIG. 5A.) Sequence Analysis Viewer (SAV) was utilized to extract the Read 1 (R1) and Read 2 (R2) intensities from the NextSeq 2000 runs. The striped black and white bar is R1 or R2 intensity of the control clustering formulation with a standard commercial recipe. The grey bar is R1 or R2 intensity using the clustering formulation supplemented with 0.3 U PPiase (pyrophosphatase) per 100 μl clustering formulation with the standard modified recipe to pull from the unique well with the cartridge. The black bar is R1 or R2 intensity using a clustering formulation supplemented with 1.2 U of PPiase per 100 μl clustering formulation with the standard modified recipe to pull from the unique well with the cartridge. For both read 1 and read 2 the presence of the PPiase increased the intensity. Additionally, the increase in intensity was observed in a concentration dependent manner meaning that in increasing the amount of enzyme utilized increases the intensity signal for both read 1 and read 2. The unit of measure of intensity is relative fluorescence units (RFU). FIG. 5B.) The Quality Score represented in the % Q30 values extracted from SAV. The control bar is the standard clustering formulation; the grey bar is the clustering formulation supplemented with 0.3 U PPiase per 100 μl clustering formulation; the black bar is the clustering formulation 1.2 U PPiase per 100 μl clustering formulation. The % Q30> scores increased in the presence of the PPiase in a concentration dependent manner relative to the control. FIG. 5C.) Instrument yield measured in G output was extracted from SAV. The control bar is the standard clustering formulation; the grey bar is the clustering formulation supplemented with 0.3 U PPiase per 100 μl clustering formulation; the black bar is the clustering formulation 1.2 U PPiase per 100 μl clustering formulation. The yield of NextSeq 2000 increased in the presence of the PPiase in a concentration dependent manner relative to the control. FIG. 5D.) Percent passing filter clusters (% PF) was extracted from SAV. The control bar is the standard clustering formulation; the grey bar is the clustering formulation supplemented with 0.3 U PPiase per 100 μl clustering formulation; the black bar is the clustering formulation 1.2 U PPiase per 100 μl clustering formulation. The % PF of NextSeq 2000 increased in the presence of the PPiase in a concentration dependent manner relative to the control. FIG. 5E.) The addition of inorganic pyrophosphatase, as a reference means to reduce to PPi.

FIG. 6 shows that PPK2 with PPi can support RPA reactions in vitro. Lanes: (M) DNA ladder with sizes noted; (1) positive control with all components of HCXE (normal amplification mixture using creatine kinase (CK) and creatine phosphate (CP); (2) HCXE+2.5 μM PPi; (3) HCXE+2.5 μM PPi+3 μM PPK2+1.8 μM ATP (CK and CP removed); (4) HCXE & 2.5 μM PPi+1.8 μM ATP (CK and CP removed); (5) negative control, template minus amplification mixture.

FIG. 7 shows a strand invasion assay (SIA) overview. An annealed duplex where one strand (red) has a black hole quencher (BHQ) attached and a strand (black) where a fluorescent dye (FAM) is attached. An oligonucleotide (oligo; green) with a region of homology to the annealed duplex (red) is present in the reaction tube. Recombinase, and combinations of the (+/−) of the energy regeneration system can be supplemented to the reaction tube. A fluorescent signal is generated when the BHQ strand is removed by the invading unlabeled strand allowing the FAM dye to be excited.

FIG. 8A.) An engineered RB32 UvsX confers thermostability to a mesophilic enzyme. A.) Alignment illustrating the amino acid differences between the engineered RB32. FIG. 8B.) SIA fluorescent plots for a range of concentrations of RB32 engineered (from 0.2 to 2 μM) at 60° C. FIG. 8C.) SIA fluorescent plots for a range of concentrations of RB32 engineered (from 0.2 to 2 μM) at 50° C. FIG. 8D.) Vmax is plotted from the fluorescent readouts calculated by the instrument software (Biotek; Cytation 5) for comparisons between the enzyme concentration. The concentrations tested are indicated in the key of the figure. The engineered RB32 UvsX has strand invasion activity at 60° C. compared to diminished activity of HQ UvsX at 60° C.

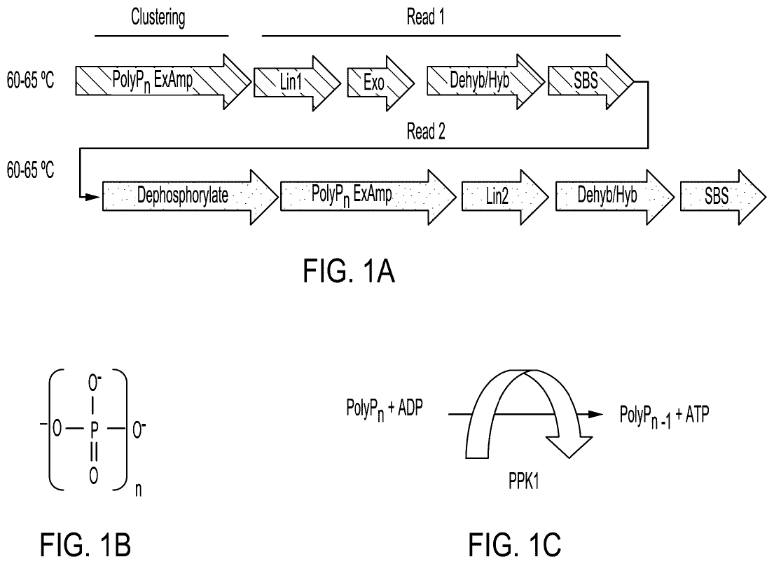

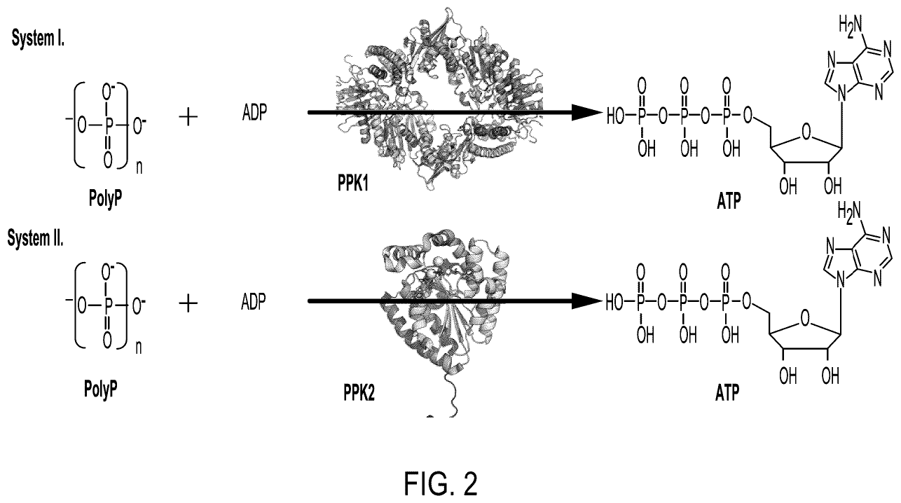

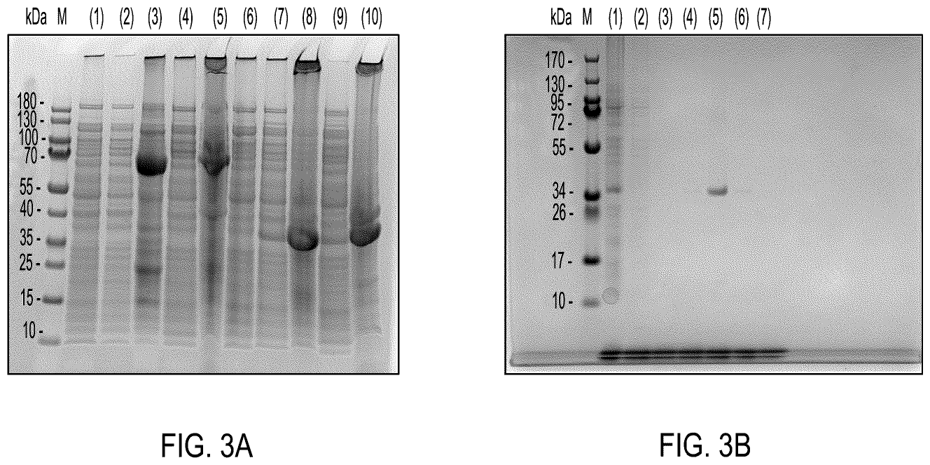

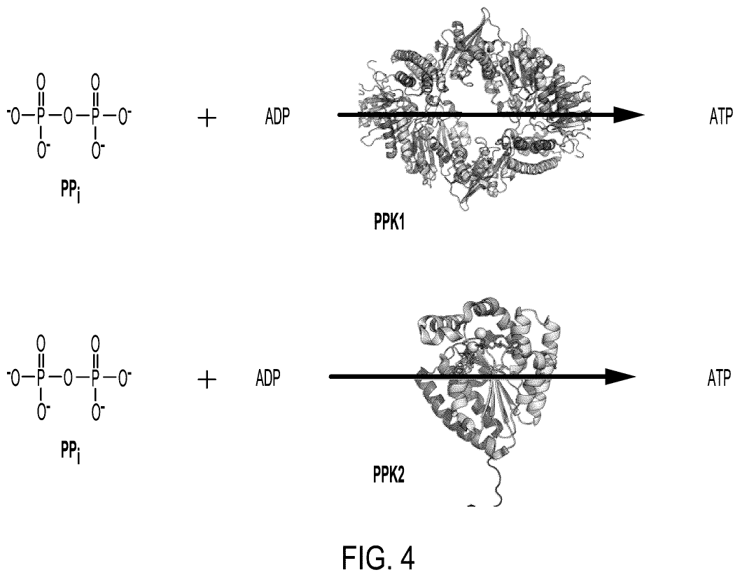

### DETAILED DESCRIPTION

The following described features apply to all aspects and embodiments of the disclosure.

The present disclosure is directed to amplification methods and compositions.

The present disclosure can be used in sequencing, for example pairwise sequencing. Methodology applicable to the present disclosure has been described in WO 08/041002, WO 07/052006, WO 98/44151, WO 00/18957, WO 02/06456, WO 07/107710, W005/068656, U.S. Ser. No. 13/661,524 and US 2012/0316086, the contents of which are herein incorporated by reference. Further information can be found in US 20060024681, US 200602926U, WO 06110855, WO 06135342, WO 03074734, W007010252, WO 07091077, WO 00179553 and WO 98/44152, the contents of which are herein incorporated by reference.

Sequencing generally comprises four fundamental steps: 1) library preparation to form a plurality of template molecules available for sequencing; 2) cluster generation to form an array of amplified single template molecules on a solid support; 3) sequencing the cluster array; and 4) data analysis to determine the target sequence.

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adapters (adapter sequences). This may be followed by amplification and sequencing. The original sample DNA fragments are referred to as “inserts”. Alternatively “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size-fractionated prior to modification with the adaptor sequences.

As used herein an “adapter” sequence comprises a short sequence-specific oligonucleotide that is ligated to the 5′ and 3′ ends of each DNA (or RNA) fragment in a sequencing library as part of library preparation. The adaptor sequence may further comprise non-peptide linkers.

As will be understood by the skilled person, a double-stranded nucleic acid will typically be formed from two complementary polynucleotide strands comprised of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

In one embodiment, the template comprises, in the 5′ to 3′ direction, a first primer-binding sequence (e.g. P5, for example, comprising the sequence as defined in SEQ ID NO: 4), an index sequence (e.g. i5), a first sequencing binding site (e.g. SBS3), an insert, a second sequencing binding site (e.g. SBS12), a second index sequence (e.g. i7) and a second primer-binding sequence (e.g. P7′, for example, comprising the sequence as defined in SEQ ID NO: 7). In another embodiment, the template comprises, in the 3′ to 5′ direction, a first primer-binding site (e.g. P5′, which is complementary to P5, for example, comprising the sequence as defined in SEQ ID NO: 6), an index sequence (e.g. i5′, which is complementary to I5), a first sequencing binding site (e.g. SBS3′ which is complementary to SBS3), an insert, a second sequencing binding site (e.g. SB S12′, which is complementary to SBS12), a second index sequence (e.g. i7′, which is complementary to I7) and a second primer-binding sequence (e.g. P7, which is complementary to P7′, for example, comprising the sequence as defined in SEQ ID NO: 5). Either template is referred to herein as a “template strand” or “a single stranded template”. Both template strands annealed together is referred to herein as “a double stranded template”.

A sequence comprising at least a primer-binding sequence (preferably a combination of a primer-binding sequence, an index sequence and a sequencing binding site) may be referred to herein as an adaptor sequence, and a single insert is flanked by a 5′ adaptor sequence and a 3′ adaptor sequence. The first primer-binding sequence may also comprising a sequencing primer for the index read (IS). “Primer-binding sequences” may also be referred to as “clustering sequences” in the present disclosure, and such terms may be used interchangeably.

The P5′ and P7′ primer-binding sequences are complementary to short primer sequences (or lawn primers) present on the surface of the flow cells. Binding of P5′ and P7′ to their complements (P5 and P7) on—for example—the surface of the flow cell, permits nucleic acid amplification. As used herein “′” denotes the complementary strand.

The primer-binding sequences in the adaptor which permit hybridisation to amplification primers (e.g. lawn primers) will typically be around 20-40 nucleotides in length, although, in embodiments, the disclosure is not limited to sequences of this length. The precise identity of the amplification primers (e.g. lawn primers), and hence the cognate sequences in the adaptors, are generally not material to the disclosure, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in W005068656, whose contents are incorporated herein by reference in their entirety. The tag can be read at the end of the first read, or equally at the end of the second read, for example using a sequencing primer complementary to the strand marked P7. The disclosure is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example U.S. 60/899221. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e. hybridises) to a portion of the sequencing binding site on the template strand. The polymerase enzyme binds to this site and incorporates complementary nucleotides base by base into the growing opposite strand. In one embodiment, the sequencing process comprises a first and second sequencing read. The first sequencing read may comprise the binding of a first sequencing primer (read 1 sequencing primer) to the first sequencing binding site (e.g. SBS3′) followed by synthesis and sequencing of the complementary strand. This leads to the sequencing of the insert. In a second step, an index sequencing primer (e.g. i7 sequencing primer) binds to a second sequencing binding site (e.g. SBS12) leading to synthesis and sequencing of the index sequence (e.g. sequencing of the i7 primer). The second sequencing read may comprise binding of an index sequencing primer (e.g. i5 sequencing primer) to the complement of the first sequencing binding site on the template (e.g. SBS3) and synthesis and sequencing of the index sequence (e.g. i5). In a second step, a second sequencing primer (read 2 sequencing primer) binds to the complement of the primer (e.g. i7 sequencing primer) binds to a second sequencing binding site (e.g. SBS12′) leading to synthesis and sequencing of the insert in the reverse direction.

Once a double stranded nucleic acid template library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al). In one embodiment, chemical denaturation is used.

Following denaturation, a single-stranded template library can be contacted in free solution onto a solid support comprising surface capture moieties (for example P5 and P7 lawn primers). This solid support is typically a flowcell, although in alternative embodiments, seeding and clustering can be conducted off-flowcell using other types of solid support.

By way of brief example, following attachment of the P5 and P7 primers to the solid support, the solid support may be contacted with the template to be amplified under conditions which permit hybridisation (or annealing—such terms may be used interchangeably) between the template and the immobilised primers. The template is usually added in free solution under suitable hybridisation conditions, which will be apparent to the skilled reader. Typically, hybridisation conditions are, for example, 5×SSC at 40° C. However, other temperatures may be used during hybridisation, for example about 50° C. to about 75° C., about 55° C. to about 70° C., or about 60° C. to about 65° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilised on the solid support and binding. Further rounds of amplification (analogous to a standard PCR reaction) lead to the formation of (monoclonal) clusters or colonies of template molecules bound to the solid support.

Thus, solid-phase amplification by either the method analogous to that of WO 98/44151 or that of WO 00/18957 (the contents of which are incorporated herein in their entirety by reference) will result in production of a clustered array comprised of colonies of “bridged” amplification products. Both strands of the amplification products will be immobilised on the solid support at or near the 5′ end, this attachment being derived from the original attachment of the amplification primers. Typically, the amplification products within each colony will be derived from amplification of a single template (target) molecule. Other amplification procedures may be used, and will be known to the skilled person. For example, amplification may be isothermal amplification using a strand displacement polymerase; or may be exclusion amplification as described in WO 2013/188582. Further information on amplification can be found in WO0206456 and WO07107710, the contents of which are incorporated herein in their entirety by reference. Through such approaches, a cluster of single template molecules is formed.

To facilitate sequencing, it is preferable if one of the strands is removed from the surface to allow efficient hybridisation of a sequencing primer to the remaining immobilised strand. Suitable methods for linearisation are described in more detail in application number WO07010251, the contents of which are incorporated herein by reference in their entirety.

Sequence data can be obtained from both ends of a template duplex by obtaining a sequence read from one strand of the template from a primer in solution, copying the strand using immobilised primers, releasing the first strand and sequencing the second, copied strand. For example, sequence data can be obtained from both ends of the immobilised duplex by a method wherein the duplex is treated to free a 3′-hydroxyl moiety that can be used an extension primer. The extension primer can then be used to read the first sequence from one strand of the template. After the first read, the strand can be extended to fully copy all the bases up to the end of the first strand. This second copy remains attached to the surface at the 5′ -end. If the first strand is removed from the surface, the sequence of the second strand can be read. This gives a sequence read from both ends of the original fragment.

Sequencing can be carried out using any suitable “sequencing-by-synthesis” technique, wherein nucleotides are added successively to the free 3′ hydroxyl group, resulting in synthesis of a polynucleotide chain in the 5′ to 3′ direction. The nature of the nucleotide added is preferably determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB/2007/001770, the contents of which are incorporated herein by reference in their entirety. Alternatively, a separate reaction may be carried out containing each of the modified nucleotides added individually.

The modified nucleotides may carry a label to facilitate their detection. In a particular embodiment, the label is a fluorescent label. Each nucleotide type may carry a different fluorescent label. However the detectable label need not be a fluorescent label. Any label can be used which allows the detection of the incorporation of the nucleotide into the DNA sequence. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the contents of which are incorporated herein by reference in their entirety.

Alternative methods of sequencing include sequencing by ligation, for example as described in U.S. Pat. No. 6,306,597 or WO06084132, the contents of which are incorporated herein by reference.

In some embodiments, sequencing may involve pairwise sequencing. The typical steps of pairwise sequencing are known and have been described in WO 2008/041002, the contents of which are herein incorporated by reference. However, the key steps will be briefly described.

Examples of the present disclosure relate to methods for sequencing two regions of a target double-stranded polynucleotide template, referred to herein as the first and second regions for sequence determination. The first and second regions for sequence determination are at both ends of complementary strands of the double-stranded polynucleotide template, which are referred to herein respectively as first and second template strands. Once the sequence of a strand is known, the sequence of its complementary strand is also known, therefore the term two regions can apply equally to both ends of a single stranded template, or both ends of a double stranded template, wherein a first region and its complement are known, and a second region and its complement are known.

A plurality of template polynucleotide duplexes are immobilised on a solid support. The template polynucleotides may be immobilised in the form of an array of amplified single template molecules, or ‘clusters’. Each of the duplexes within a particular cluster comprises the same double-stranded target region to be sequenced. The duplexes are each formed from complementary first and second template strands which are linked to the solid support at or near to their 5′ ends. Typically, the template polynucleotide duplexes will be provided in the form of a clustered array.

An alternate starting point is a plurality of single stranded templates which are attached to the same surface as a plurality of primers that are complementary to the 3′ end of the immobilised template. The primers may be reversibly blocked to prevent or inhibit extension. The single stranded templates may be sequenced using a hybridised primer at the 3′ end. The sequencing primer may be removed after sequencing, and the immobilised primers deblocked to release an extendable 3′ hydroxyl. These primers may be used to copy the template using bridged strand resynthesis to produce a second immobilised template that is complementary to the first. Removal of the first template from the surface allows the newly single stranded second template to be sequenced, again from the 3′ end. Thus, both ends of the original immobilised template can be sequenced. Such a technique allows paired end reads where the templates are amplified using a single extendable immobilised primer, for example as described in Polony technology (Nucleic Acids Research 27, 24, e34(1999)) or emulsion PCR (Science 309, 5741, 1728-1732 (2005); Nature 437, 376-380 (2005)).

As provided herein, in nonlimiting examples of the present disclosure, changing the energy recycling system used in amplification systems for sequencing enables better tuning of the kinetics of the amplification reactions used and thereby allowing higher monoclonality to be obtained in clusters, as compared to energy recycling systems used previously in amplification, for example creatine kinase/creatine phosphate systems. This in turn leads to higher quality sequencing results, and thereby increased throughput of sequencing results.

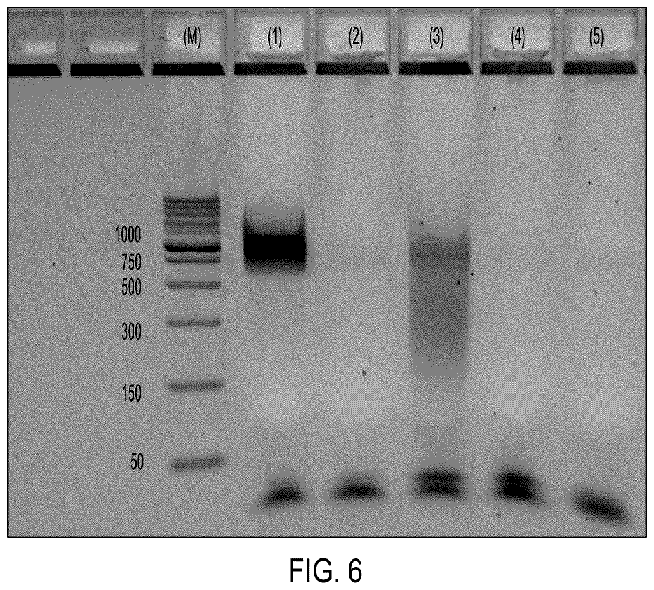

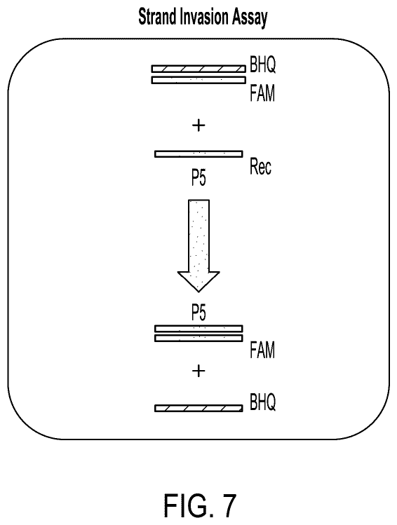

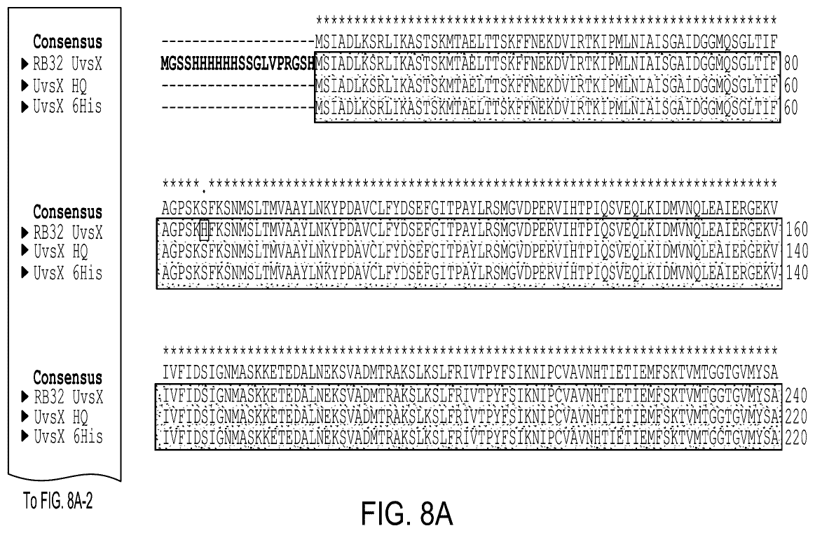

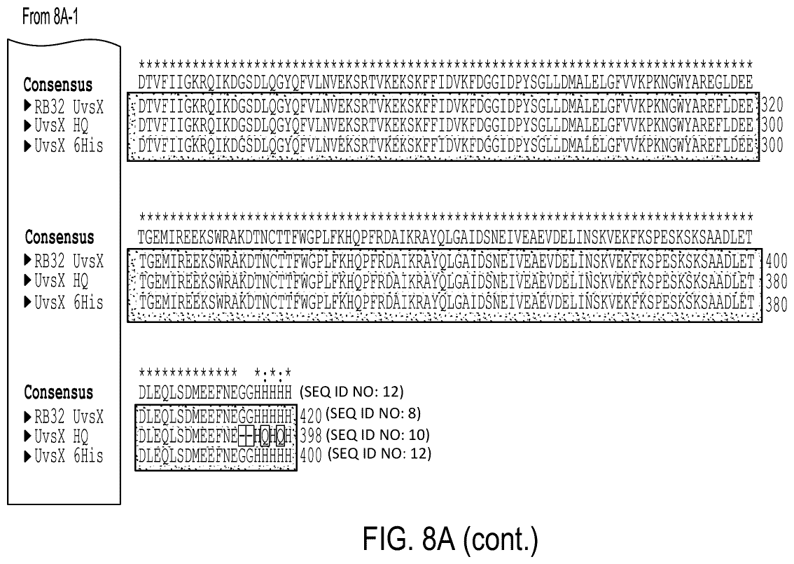

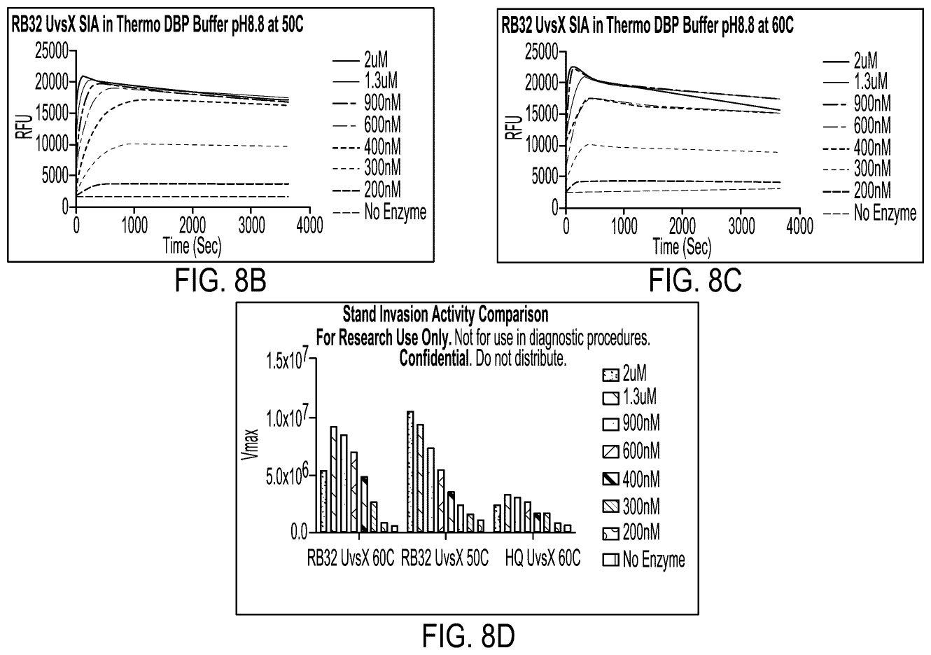

As shown in FIG. 6, the present inventors have found that the generation of ATP during amplification by the polyphosphate kinase, provides a source of ATP for use by the recombinase to perform homology searching and strand invasion.

The present inventors have also found that the removal of inorganic pyrophosphate from the amplification composition, for example by the addition of polyphosphate kinase, has a number of advantages in methods of nucleic acid amplification and sequencing. Specifically, the present inventors have found that the removal of inorganic pyrophosphate can be used to improve clustering kinetics, and in turn reduce clustering times (and thus turnaround times) and/or increase the signal intensities (and thus increase the sequence signal:noise ratios).

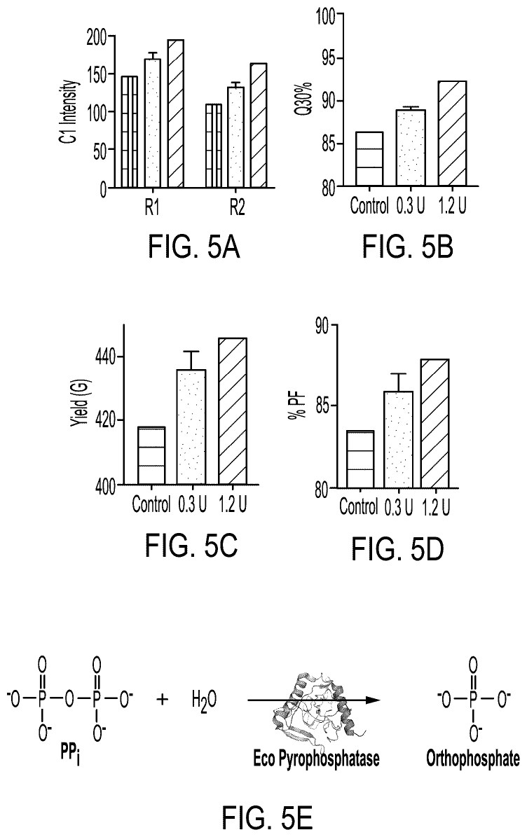

Specifically, the present inventors have found that improving clustering kinetics by the removal or reduction of PPi leads to improvements in sequencing performance, including, but not limited to, an increase in intensity, % PF, Q30 and Yield (g). By “% PF” is meant the % of reads that pass the chastity filter (chastity is the ratio is the ratio of the brightest base intensity divided by the sum of the brightest and second brightest base intensities”). By “Q30” is meant the percentage of bases with a quality score of 30 or higher. A quality score is “A quality score is an estimate of the probability of that base being called wrongly: q=−10×log10(p)”. By “yield” is meant the number of bases generated in the run. This is shown in FIG. 5. Here the addition of inorganic pyrophosphatase, as a reference means to reduce to PPi (as shown in FIG. 5E), at 0.3 U and 1.2 U increasing the intensity (FIG. 5A), % PF (FIG. 5B), Q30 (FIG. 5C) and Yield (g)(FIG. 5D).

In addition, increasing clustering intensity also allows amplification/clustering to take place in smaller wells, where a decrease in well size requires an increase in signal intensity.

Furthermore, as explained above, the accumulation of inorganic pyrophosphate stalls DNA polymerase. This is problematic where the DNA polymerase encounters structured secondary features like a G-quadruplex, leading to parts of the library that are not clustered/amplified and therefore not sequenced. Removal of inorganic pyrophosphate prevents or inhibits stalling of the DNA polymerase, and consequently a decrease in sequence specific errors because the polymerase is able to cluster/amplify structured regions of the genome.

Finally, in addition to improving clustering kinetics (e.g. clustering times and the signal intensity), the addition of polyphosphate kinase can also significantly reduce the amount of clustering/amplification reagents needed by as much as 50%. As mentioned, in an amplification or clustering reaction it may be necessary to add the amplification composition more than once (the number of times the amplification composition is added to the flowcell may be called a “push”). Multiple pushes may be necessary to achieve the required level of sequence signal intensity. The removal of inorganic pyrophosphate can significantly increase the sequence signal intensity with a single push. Accordingly, by reducing PPi levels it is possible to additionally half the amount of amplification composition needed (i.e. half the COGs (cost of goods) without affecting clustering/amplification intensities.

As used herein, the term “cluster” may refer to a clonal group of template polynucleotides (e.g. DNA or RNA) bound within a single well of a flowcell. A “cluster” may contain a sufficient number of copies of a single template polynucleotide such that the cluster is able to output a signal (e.g. a light signal) that allows a single sequencing read to be performed on the cluster. A “cluster” may comprise, for example, about 500 to about 2000 copies, preferably about 600 to about 1800 copies, more preferably about 700 to about 1600 copies, even more preferably about 800 to 1400 copies, yet even more preferably about 900 to 1200 copies, most preferably about 1000 copies of a single template polynucleotide. The copies of the single template polynucleotide may comprise at least about 50%, preferably at least about 60%, more preferably at least about 70%, even more preferably at least about 80%, yet even more preferably at least about 90%, most preferably about 95%, 98%, 99% or 100% of all polynucleotides within a single well of the flowcell, and thus providing a substantially monoclonal “cluster”.

In an embodiment, examples of the present disclosure are directed to an amplification composition comprising an inorganic polyphosphate and a polyphosphate kinase.

As used herein, the term “inorganic polyphosphate” may refer to a system of two or more phosphate residues connected by phosphoanhydride bonds. The system may be linear.

An inorganic polyphosphate may be present in an acid form, a salt form, or a combination thereof. In cases where the inorganic polyphosphate is present in a salt form, the inorganic polyphosphate may comprise a cation (not including H⁺). For example, the cation may be selected from “metal cations” or “non-metal cations”. Metal cations may include alkali metal ions (e.g. lithium, sodium, potassium, rubidium or caesium ions). Non-metal cations may include ammonium salts (e.g. alkylammonium salts) or phosphonium salts (e.g. alkylphosphonium salts).

The inorganic polyphosphate may be soluble in aqueous medium.

As used herein, the term “polyphosphate kinase” may refer to an enzyme which catalyses the following reaction:

PolyPₙ+ADP→PolyPₙ₋₁+ATP

wherein PolyPₙ refers to an inorganic polyphosphate with “n” phosphate residues, ADP refers to adenosine diphosphate, PolyPₙ₋₁ refers to an inorganic polyphosphate with “n−1” phosphate residues, and ATP refers to adenosine triphosphate.

The composition may comprise the inorganic polyphosphate at a concentration of about 0.01 μM to about 1000 about 0.1 μM to about 100 about 0.5 μM to about 50 about 1 μM to about 20 or about 2 μM to about 10 μM. Alternatively, the inorganic polyphosphate is present at a wt % between about 0.01 wt % to about 5.0 wt %, about 0.02 wt % to about 4.5 wt %, about 0.05 wt % to about 4.0 wt %, about 0.08 wt % to about 3.5 wt %, about 0.1 wt % to about 3.0 wt %, about 0.2 wt % to about 2.5 wt %, or about 0.5 wt % to about 2.0 wt % with respect to a total wt % of the composition by dry mass.

The composition may comprise the polyphosphate kinase at a concentration of about 0.01 μM to about 1000 μM about 0.1 μM to about 100 about 0.5 μM to about 50 about 1 μM to about 20 or about 2μM to about 10 μM. Alternatively, the composition comprises between about 0.01 U/μL and about 100 U/μL of the polyphosphate kinase, between about 0.1 U/μL and about 50 U/μL, between about 0.2 U/μL and about 30 U/μL, between about 0.3 U/μL and about 20 U/μL, between about 0.5 U/μL and about 10 U/μL, or between about 1.0 U/μL and about 5.0 U/μL. For example, the composition may comprise around 0.3 U/μL, 0.4 U/μL, 0.5 U/μL, 0.6 U/μL, 0.7 U/μL, 0.8 U/μL, 0.9 U/μL, 1.0 U/μL, 1.1 U/μL, 1.2 U/μL, 1.3 U/μL, 1.4 U/μL, 1.5 U/μL, 1.6 U/μL, 1.7 U/μL, 1.8 U/μL, 1.9 U/μL or around 2.0 U/μL of the polyphosphate kinase. Alternatively, the polyphosphate kinase is present at a wt % between about 0.01 wt % to about 5.0 wt %, about 0.02 wt % to about 4.5 wt %, about 0.05 wt % to about 4.0 wt %, about 0.08 wt % to about 3.5 wt %, about 0.1 wt % to about 3.0 wt %, about 0.2 wt % to about 2.5 wt %, or about 0.5 wt % to about 2.0 wt % with respect to a total wt % of the composition by dry mass.

The inorganic polyphosphate may comprise a first inorganic polyphosphate with less than 50 phosphate residues. For example, the first inorganic polyphosphate may comprise 2 to 50 phosphate residues, preferably 5 to 45 phosphate residues, more preferably 10 to 40 phosphate residues, even more preferably 15 to 35 phosphate residues. In a preferred embodiment, the first inorganic polyphosphate may be pyrophosphate (two phosphate residues). The number of phosphate residues may refer to an average number of phosphate residues (e.g. a median number of phosphate residues).

The inorganic polyphosphate may comprise a second inorganic polyphosphate with more than 100 phosphate residues. For example, the second inorganic polyphosphate may comprise 100 to 10000 phosphate residues, preferably 150 to 5000 phosphate residues, more preferably 200 to 2000 phosphate residues, even more preferably 250 to 1000 phosphate residues. The number of phosphate residues may refer to an average number of phosphate residues (e.g. a median number of phosphate residues).

A ratio of the first inorganic polyphosphate to the second inorganic polyphosphate may be about 99:1 to about 1:99, about 98:2 to about 2:98, about 95:5 to about 5:95, about 90:10 to about 10:90, about 85:15 to about 15:85, about 80:20 to about 20:80, about 75:25 to about 25:75, about 70:30 to about 30:70, about 65:35 to about 35:65, about 60:40 to about 40:60, about 55:45 to about 45:55, or about 50:50. In some embodiments (e.g. for a first polyphosphate composition as described herein), a ratio of the first inorganic polyphosphate to the second inorganic polyphosphate is about 99:1 to about 50:50, preferably about 98:2 to about 55:45, more preferably about 95:5 to about 60:40, even more preferably about 90:10 to about 65:35, yet even more preferably about 85:15 to about 70:30, most preferably about 80:20 to about 75:25. In some embodiments (e.g. for a second polyphosphate composition as described herein), a ratio of the first inorganic polyphosphate to the second inorganic polyphosphate is about 50:50 to about 1:99, preferably about 45:55 to about 2:98, more preferably about 40:60 to about 5:95, even more preferably about 35:65 to about 10:90, yet even more preferably about 30:70 to about 15:85, most preferably about 25:75 to about 20:80.

The polyphosphate kinase may be selected from the group consisting of: a polyphosphate kinase of the PPK1 family, a polyphosphate kinase of the PPK2 family, and a polyphosphate kinase of the PPK3 family. Preferably, the polyphosphate kinase is selected from the group consisting of: a polyphosphate kinase of the PPK1 family, and a polyphosphate kinase of the PPK2 family.

The polyphosphate kinase may be a thermophilic or a mesophilic polyphosphate kinase.

In one embodiment, the polyphosphate kinase is derived from a thermophile (including a hyperthermophile). Examples of thermophiles or hyperthermophile include microbes from the family Thermococcaceae, Thermaceae or Thermotogaceae; or from the genus Thermus, the genus Meiothermus, the genus Thermococcus, the genus Pyrococcus or the genus Thermotoga. In one embodiment, the thermophile may be selected from Thermococcus kodacaraensis, Meiothermus ruber, Pyrococcus abyssi, Pyrococcus furiosus, Pyrococcus species GB-D, Pyrococcus woesei, Thermus aquaticus, Thermus brokianus, Thermus caldophilus, Thermus filiformis, Thermus flavus, Thermococcus fitmiculans, Thermococcus gorgonarius, Thermococcus litoralis, Thermotoga maritima, Thermotoga neopolitana and Thermus thermophilus.

In one embodiment, the thermophile is from the genus Thermus. In one embodiment, the thermophile is Thermus thermophilus and the polyphosphate kinase may comprise the following sequence selected from the following, or a functional variant or functional fragment thereof.

(SEQ ID NO: 1)
HLLPEASWLQFNRRVLLQTERPDFPLLERLRFLGIWNRNL
DEFFAARIAKPFLKSRRGPDHLALLQEALDQAKLARARYQ
NLLQEAFPRLRVLDPGELDDLDWLYFRVFLAEEVAPKTDL
IPWEAAQDLSHSALYFASERYLVRLPQDLPRLVEVPGREG
TYVRLGALMRWRSDLLLPEEAPLYEFRVLRLLESERVRAD
WNELAESLEGRQEGTPTLLVVEEGFPEAWLDALRRALGLF
LEEVFALKPPLNLSLVDTLVAQGPPEWRFPPFRPERPRTF
LKNPLALLGKRDVLLYHPFEDYAAVERFAEAALAEEVEEV
WATLYRTGEENPLAEALIAAARKGKRVHVLLEGRARFDEL
LNLRWYLRLVRAGVEVLPLPERKVHAKAFLILTREGGYAH
LGTGNYNPTNGHHYTDFSLFTARKEVVAEVRAFFQAMAEE
KTPRLGLLRTGEGIRRLLLEAVLHEAHPKGRLILKFNHLT
DPELLEALVYAASRGARVDLLVRSTLTRLHPAIRAKSLVG
RFLEHARAAAFRAGGEWRVYLTSADAMPRNFQNRFELLFP
VLDKEAKKKVLKVLKRQVRDDRNSFLLTPEGEKRLWGGRH
DAQRL.

In one embodiment, the thermophile is from the genus Meiothermus. In one embodiment, the thermophile is Meiothermus ruber and the polyphosphate kinase may comprise the following sequence, or a functional variant or functional fragment thereof:

(SEQ ID NO: 2)
KKYRVQPDGRFELKRFDPDDTSAFEGGKQAALEALAVLNR
RLEKLQELLYAEGQHKVLVVLQAMDAGGKDGTIRVVFDGV
NPSGVRVASFGVPTEQELARDYLWRVHQQVPRKGELVIFN
RSHYEDVLVVRVKNLVPQQVWQKRYRHIREFERMLADEGT
TILKFFLHISKDEQRQRLQERLDNPEKRWKFRMGDLEDRR
LWDRYQEAYEAAIRETSTEYAPWYVIPANKNWYRNWLVSH
ILVETLEGLAMQYPQPETASEKIVIE.

In one embodiment, the polyphosphate kinase is derived from a mesophile. Examples of a mesophile include Saccharomyces cerevisiae and E. coli. In one embodiment, the polyphosphate kinase comprises the sequence as shown in SEQ ID NO: 3 or a functional variant or functional fragment thereof:

(SEQ ID NO: 3)
MGQEKLYIEKELSWLSFNERVLQEAADKSNPLIERMRFLG
IYSNNLDEFYKVRFAELKRRIIISEEQGSNSHSRHLLGKI
QSRVLKADQEFDGLYNELLLEMARNQIFLINERQLSVNQQ
NWLRHYFKQYLRQHITPILINPDTDLVQFLKDDYTYLAVE
IIRGDTIRYALLEIPSDKVPRFVNLPPEAPRRRKPMILLD
NILRYCLDDIFKGFFDYDALNAYSMKMTRDAEYDLVHEME
ASLMELMSSSLKQRLTAEPVRFVYQRDMPNALVEVLREKL
TISRYDSIVPGGRYHNFKDFINFPNVGKANLVNKPLPRLR
HIWFDKAQFRNGFDAIRERDVLLYYPYHTFEHVLELLRQA
SFDPSVLAIKINIYRVAKDSRIIDSMIHAAHNGKKVTVVV
ELQARFDEEANIHWAKRLTEAGVHVIFSAPGLKIHAKLFL
ISRKENGEVVRYAHIGTGNFNEKTARLYTDYSLLTADARI
TNEVRRVFNFIENPYRPVTFDYLMVSPQNSRRLLYEMVDR
EIANAQQGLPSGITLKLNNLVDKGLVDRLYAASSSGVPVN
LLVRGMCSLIPNLEGISDNIRAISIVDRYLEHDRVYIFEN
GGDKKVYLSSADWMTRNIDYRIEVATPLLDPRLKQRVLDI
IDILFSDTVKARYIDKELSNRYVPRGNRRKVRAQLAIYDY
IKSLEQPE.

As used herein, the term “thermophilic” or “thermostable” may refer to a protein that does not substantially denature at high temperature, for example above 40° C., above 45° C., above 50° C., above 55° C., above 60° C., above 65° C., above 70° C., above 75° C., above 80° C., above 85° C., above 90° C., above 95° C., or above 100° C.

The polyphosphate kinase may have an optimum working temperature of about 50° C. to about 75° C., preferably about 55° C. to about 70° C., or more preferably about 60° C. to about 65 ° C.; for example, the polyphosphate kinase may have an optimum working temperature of about 50° C., about 55° C., about 60° C., about 65° C., about 70° C., or about 75° C.

As used herein, the term “optimum working temperature” may refer to a temperature at which the catalytic activity of the enzyme reaches a peak maximum value.

As used herein, the term “functional variant” refers to a variant polypeptide sequence or part of the polypeptide sequence which retains the biological function of the full non-variant sequence. For example, a functional variant of polyphosphate kinase is able to catalyse the conversion of inorganic polyphosphate and ADP to ATP.

A functional variant also comprises a variant of the polypeptide of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a polypeptide sequence that does not affect the functional properties of the polypeptide are well known in the art. For example, the amino acid alanine, a hydrophobic amino acid, may be substituted by another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Each of the proposed modifications is well within the routine skill in the art, as is determination of retention of biological activity of the encoded products.

As used in any aspect described herein, a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant amino acid sequence and preferably retains the catalytic activity of a polyphosphate kinase as described above. The sequence identity of a variant can be determined using any number of sequence alignment programs known in the art. As an example, Emboss Stretcher from the EMBL-EBI may be used: https://www.ebi.ac.uk/Tools/psa/emboss_stretcher/ (using default parameters: pair output format, Matrix=BLOSUM62, Gap open=1, Gap extend=1 for proteins; pair output format, Matrix=DNAfull, Gap open=16, Gap extend=4 for nucleotides).

As used herein, the term “functional fragment” refers to a functionally active series of consecutive amino acids from a longer polypeptide or protein. For example, a functional fragment may retain the catalytic activity of a polyphosphate kinase, as described above.

The composition may further comprise a recombinase. The recombinase may be a thermophilic recombinase.

As used herein, the term “recombinase” may refer to an enzyme which can facilitate invasion of a target nucleic acid by a polymerase and extension of a primer by the polymerase using the target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g. via heating or chemical denaturation) is not required. As such, recombinase-facilitated amplification can be carried out isothermally. It is generally desirable to include ATP, or other nucleotides (or in some cases non-hydrolysable analogs thereof) in a recombinase-facilitated amplification reagent to facilitate amplification. A mixture of recombinase and single-stranded binding (SSB) protein is particularly useful as SSB can further facilitate amplification. Recombinases may include, for example, RecA protein, the T4 uvsX protein, any homologous protein or protein complex from any phyla, or functional variants thereof. Eukaryotic RecA homologues are generally named Rad51 after the first member of this group to be identified. Other non-homologous recombinases may be utilised in place of RecA, for example, RecT or RecO.

In some preferred embodiments, the recombinase may be UvsX. In one embodiment, the UvsX comprises or consists of SEQ ID NO: 8 or 9 or a functional fragment or functional variant thereof.

In other preferred embodiments, the recombinase may be a thermophilic UvsX. In one embodiment, the thermophilic UvsX comprises or consists of SEQ ID NO: 10 or 11 or a functional fragment or functional variant thereof.

The composition may further comprise a single-stranded nucleotide binding protein.

As used herein, the term “single-stranded nucleotide binding protein” may refer to any protein having a function of binding to a single stranded nucleic acid, for example, to prevent or inhibit premature annealing, to protect the single-stranded nucleic acid from nuclease digestion, to remove secondary structure from the nucleic acid, or to facilitate replication of the nucleic acid. The term is intended to include, but is not necessarily limited to, proteins that are formally identified as Single Stranded Binding proteins by the Nomenclature Committee of the International Union of Biochemistry and Molecular Biology (NC-IUBMB). Exemplary single stranded binding proteins include, but are not limited to E. coli SSB, T4 gp32, T7 gene 2.5 SSB, phage phi 29 SSB, any homologous protein or protein complex from any phyla, or functional variants thereof.

The composition may further comprise a polymerase. Preferably, the polymerase may be a strand-displacing polymerase. In some preferred embodiments, the polymerase may be a DNA polymerase. In other preferred embodiments, the polymerase may be a RNA polymerase. The polymerase may be a thermophilic polymerase.

As used herein, the term “polymerase” may refer to an enzyme that produces a complementary replicate of a nucleic acid molecule using the nucleic acid as a template strand. Typically, DNA polymerases bind to the template strand and then move down the template strand sequentially adding nucleotides to the free hydroxyl group at the 3′ end of a growing strand of nucleic acid. DNA polymerases typically synthesise complementary DNA molecules from DNA templates and RNA polymerases typically synthesise RNA molecules from DNA templates (transcription). Polymerases can use a short RNA or DNA strand, called a primer, to begin strand growth. Some polymerases can displace the strand upstream of the site where they are adding bases to a chain. Such polymerases are said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

The composition may further comprise a nucleotide triphosphate (NTP). Preferably, the nucleotide triphosphate may be a deoxynucleotide triphosphate (dNTP). More preferably, the composition comprises a plurality of NTPs or dNTPs, and preferably a mixture—for example comprising a plurality of dATP, dGTP, dCTP and dTTP for DNA clustering/synthesis or ATP, GTP, CTP and UTP for RNA clustering/synthesis. In one embodiment, the concentration of dNTPs may be between 0.1 and 2 mM, preferably between 0.2 to 1.5 mM, more preferably between 0.3 to 1.2 mM, even more preferably between 0.3 to 0.6 mM; for example, the concentration may be selected from 0.3 mM, 0.6 mM and 1.2 mM.

As used herein, the term “nucleotide triphosphate” may refer to a molecule containing a nitrogenous base (e.g. adenine, thymine, cytosine, guanine, uracil) bound to a 5-carbon sugar (e.g. ribose or deoxyribose), with three phosphate groups bound to the sugar.

As used herein, the term “deoxynucleotide triphosphate” or (dNTPs) may refer to a molecule containing a nitrogenous base (e.g. adenine, thymine, cytosine, guanine, uracil) bound to deoxyribose, with three phosphate groups bound to the deoxyribose.

In some embodiments, the composition may not comprise creatine kinase and/or creatine phosphate.

The composition may comprise an inorganic polyphosphate, a polyphosphate kinase, and at least one selected from the group consisting of: a recombinase, a single-stranded nucleotide binding protein, a polymerase, nucleotide triphosphates (NTPs). Preferably, the composition may comprise an inorganic polyphosphate, a polyphosphate kinase, and at least two selected from the group consisting of: a recombinase, a single-stranded nucleotide binding protein, a polymerase, nucleotide triphosphates (NTPs). More preferably, the composition may comprise an inorganic polyphosphate, a polyphosphate kinase, and at least three selected from the group consisting of: a recombinase, a single-stranded nucleotide binding protein, a polymerase, nucleotide triphosphates (NTPs).

Preferably, the composition further comprises at least one selected from the group comprising a recombinase, NTPs and a single stranded nucleotide binding (SSB) protein. More preferably, the composition further comprises at least two selected from the group comprising a recombinase, NTPs and a single stranded nucleotide binding (SSB) protein.

Preferably, the composition may further comprise a recombinase, NTPs and a single stranded nucleotide binding (SSB) protein.

Preferably, the composition may comprise an inorganic polyphosphate, a polyphosphate kinase, a recombinase, a single-stranded nucleotide binding protein, a polymerase, and nucleotide triphosphates (NTPs).

In some embodiments, the composition may not comprise one or more primers, either an amplification or a sequencing primer. Accordingly, the composition may not comprise primers. That is, the composition may not comprise any nucleic acid sequences that can initiate DNA synthesis (by a polymerase). The primers may be free nucleic acid sequence of between 18 and 22 base pairs, more preferably between 15 to 30 base pairs. The GC content of the free nucleic acid sequence may also be between 50 and 55%, and preferably, may have a GC-lock (a G or C in the last 5 bases of the sequence) at the 3′ end. The melting temperature of the primers may be between 40 and 60° C., more preferably between 50 and 55° C. The primers may also be complementary or substantially complementary (with e.g. at least 80% overall sequence identity) to a target sequence or complement thereof that the composition is intended to cluster. The primers may also comprise one or more restriction sites.

In some embodiments, the composition may also comprise a nucleic acid template. The nucleic acid template may also comprise the adaptor sequences described herein, where preferably the adaptor sequences comprise at least one of P5, P5′, P7 and P7′, the sequences of which are described below.

The composition may not comprise PEG.

The composition may comprise a buffer. Preferably, the amplification composition is buffered to a pH of about 6.0 to about 9.0, preferably about 6.5 to about 8.8, more preferably about 7.5 to about 8.7, even more preferably about 8.3 to about 8.6.

The composition may be supplied in a dry form (e.g. a freeze-dried form). In such a case, the amplification composition may be rehydrated, for example with water or a buffer solution, prior to use in amplification. In other embodiments, the amplification composition may be supplied as a solution (e.g. as an aqueous solution).

The composition may further comprise excipients. Suitable excipients may include surfactants, such as anionic surfactants, including alkyl sulfates (e.g. ammonium lauryl sulfate, sodium lauryl sulfate, sodium laureth sulfate, sodium myreth sulfate, sodium docusate), alkyl sulfonates (e.g. perfluorooctanesulfonate, perfluorobutanesulfonate), alkyl phosphates (e.g. alkyl-aryl ether phosphates, alkyl ether phosphates) and alkyl carboxylates (e.g. sodium stearate, sodium lauroyl sarcosinate, perfluorononanoate, perfluorooctanoate); cationic surfactants, including quaternary ammonium salts (e.g. cetrimonium bromide, cetylpyridinium chloride, benzalkonium chloride, benzethonium chloride, dimethyldioctadecylammonium chloride, dioctadecyldimethylammonium bromide); non-ionic surfactants, including fatty alcohol ethoxylates, alkylphenol ethoxylates, fatty acid ethoxylates, ethoxylated amines or fatty acid amides, poloxamers, polysorbates, (e.g. polyethylene glycol sorbitan alkyl esters (Tween)). Further excipients may include enzyme stabilisers, such as dithiothreitol (DTT), tris(2-carboxyethyl)phosphine (TCEP) and 2-mercaptoethanol (BME). Still further excipients may include molecular crowding agents such as polyethylene glycol (PEG), dextrans and epichlorohydrin-sucrose polymers (e.g. Ficoll); in some embodiments, PEG may be excluded.

Preferably, the amplification composition may be a clustering composition.

By “amplification composition” is meant a composition that is suitable for the amplification of a target nucleic acid template.

By contrast, a “clustering composition” refers to a composition that is suitable for the amplification of a (single) target sequence into a cluster (i.e. the composition is suitable for cluster generation, particularly for the generation of a monoclonal cluster) as described above, not just for any amplification method. In one embodiment, the composition is not additionally suitable for the detection or sequencing of the nucleic acid template. For example, in one embodiment, the composition does not comprise a fluorescent entity, such as probes, nucleotides labelled with a fluorescent entity, and/or primers labelled with a fluorescent entity. Alternatively, the composition does not comprise leuco dyes/reagents labelled with leuco dyes.

In one embodiment, the composition may be a resynthesis composition. By resynthesis is meant the step between the first and second sequencing reads where the template is copied using bridged strand resynthesis to produce a second immobilised template that is complementary to the first. Accordingly, the same composition as described herein may be used in resynthesis.

In one embodiment, the composition may be a sequencing-by-synthesis amplification composition.

In a further embodiment, examples of the present disclosure are directed to a kit comprising an inorganic polyphosphate and a polyphosphate kinase. The polyphosphate kinase may be provided separately from the inorganic polyphosphate. For example, the polyphosphate kinase may be in a different container to the inorganic polyphosphate.

Preferably, the kit may comprise an amplification composition as described herein.

The kit may further comprise a recombinase as described herein. The recombinase may be provided separately from the inorganic polyphosphate and/or the polyphosphate kinase (e.g. separately from the amplification composition as described herein). For example, the recombinase may be in a different container to the inorganic polyphosphate and/or the polyphosphate kinase (e.g. a different container to the amplification composition as described herein).

The kit may further comprise a single-stranded nucleotide binding protein as described herein. The single-stranded nucleotide binding protein may be provided separately from the inorganic polyphosphate and/or the polyphosphate kinase (e.g. separately from the amplification composition as described herein). For example, the single-stranded nucleotide binding protein may be in a different container to the inorganic polyphosphate and/or the polyphosphate kinase (e.g. a different container to the amplification composition as described herein).

The kit may further comprise a polymerase as described herein. The polymerase may be provided separately from the inorganic polyphosphate and/or the polyphosphate kinase (e.g. separately from the amplification composition as described herein). For example, the polymerase may be in a different container to the inorganic polyphosphate and/or the polyphosphate kinase (e.g. a different container to the amplification composition as described herein).

The kit may further comprise a plurality and mixture of nucleotide triphosphate (NTPs) as described herein. The nucleotide triphosphate may be provided separately from the inorganic polyphosphate and/or the polyphosphate kinase (e.g. separately from the amplification composition as described herein). For example, the nucleotide triphosphate may be in a different container to the inorganic polyphosphate and/or the polyphosphate kinase (e.g. a different container to the amplification composition as described herein).

The kit may comprise an inorganic polyphosphate, a polyphosphate kinase, and at least one selected from the group consisting of: a recombinase, a single-stranded nucleotide binding protein, a polymerase, and nucleotide triphosphates (NTPs). Preferably, the kit may comprise an inorganic polyphosphate, a polyphosphate kinase, and at least two selected from the group consisting of: a recombinase, a single-stranded nucleotide binding protein, a polymerase, and nucleotide triphosphates (NTPs). More preferably, the kit may comprise an inorganic polyphosphate, a polyphosphate kinase, and at least three selected from the group consisting of: a recombinase, a single-stranded nucleotide binding protein, a polymerase, and nucleotide triphosphates (NTPs). One or more (e.g. each of these components) may be provided separately from the inorganic polyphosphate and/or the polyphosphate kinase (e.g. separately from the amplification composition as described herein). For example, one or more (e.g. each of these components) may be in a different container to the inorganic polyphosphate and/or the polyphosphate kinase (e.g. a different container to the amplification composition as described herein).

Preferably, the kit further comprises at least one selected from the group comprising a recombinase, NTPs and a single stranded nucleotide binding (SSB) protein. More preferably, the composition further comprises at least two selected from the group comprising a recombinase, NTPs and a single stranded nucleotide binding (SSB) protein. One or more (e.g. each of these components) may be provided separately from the inorganic polyphosphate and/or the polyphosphate kinase (e.g. separately from the amplification composition as described herein). For example, one or more (e.g. each of these components) may be in a different container to the inorganic polyphosphate and/or the polyphosphate kinase (e.g. a different container to the amplification composition as described herein).

Preferably, the kit may comprise an inorganic polyphosphate, a polyphosphate kinase, a recombinase, NTPs and a single stranded nucleotide binding (SSB) protein. One or more (e.g. each of these components) may be provided separately from the inorganic polyphosphate and/or the polyphosphate kinase (e.g. separately from the amplification composition as described herein). For example, one or more (e.g. each of these components) may be in a different container to the inorganic polyphosphate and/or the polyphosphate kinase (e.g. a different container to the amplification composition as described herein).

Preferably, the kit may comprise an inorganic polyphosphate, a polyphosphate kinase, a recombinase, a single-stranded nucleotide binding protein, a polymerase, and nucleotide triphosphates (NTPs). One or more (e.g. each of these components) may be provided separately from the inorganic polyphosphate and/or the polyphosphate kinase (e.g. separately from the amplification composition as described herein). For example, one or more (e.g. each of these components) may be in a different container to the inorganic polyphosphate and/or the polyphosphate kinase (e.g. a different container to the amplification composition as described herein).

The kit may further comprise excipients as described herein. The excipient(s) may be provided separately from the inorganic polyphosphate and/or the polyphosphate kinase (e.g. separately from the amplification composition as described herein). For example, the excipient(s) may be in a different container to the inorganic polyphosphate and/or the polyphosphate kinase (e.g. a different container to the amplification composition as described herein).

The kit may further comprise one or more agents for use in preparing a template nucleic acid sequence for clustering and sequencing (i.e. library preparation agents). In one embodiment, the kit may further comprise adaptor sequences. The adaptor sequences may be configured such that they can be ligated onto a nucleic acid template to be sequenced. In some preferred embodiments, the kit may comprise a first adaptor sequence that comprises a sequence according to SEQ ID NO. 4 (P5) or a variant or fragment thereof In other preferred embodiments, the kit may comprise a second adaptor sequence that comprises a sequence according to SEQ ID NO. 5 (P7) or a variant or fragment thereof. In other preferred embodiments, the kit may comprise a third adaptor sequence that comprises a sequence according to SEQ ID NO. 6 (P5′) or a variant or fragment thereof. In other preferred embodiments, the kit may comprise a fourth adaptor sequence that comprises a sequence according to SEQ ID NO. 7 (P7′) or a variant or fragment thereof. More preferably, the kit may comprise at least two of the group selected from the first adaptor sequence, the second adaptor sequence, the third adaptor sequence and the fourth adaptor sequence. Even more preferably, the kit may comprise at least three of the group selected from the first adaptor sequence, the second adaptor sequence, the third adaptor sequence and the fourth adaptor sequence. Yet even more preferably, the kit may comprise the first adaptor sequence, the second adaptor sequence, the third adaptor sequence and the fourth adaptor sequence. The adaptor sequence(s) (e.g. each of the adaptor sequence(s)) may be provided separately from the inorganic polyphosphate and/or the polyphosphate kinase (e.g. separately from the amplification composition as described herein). For example, the adaptor sequence(s) (e.g. each of the adaptor sequence(s)) may be in a different container to the inorganic polyphosphate and/or the polyphosphate kinase (e.g. a different container to the amplification composition as described herein).The kit may further comprise a metal cofactor composition. The metal cofactor may be configured to activate one or more enzymes in the amplification composition. For example, the metal cofactor may be configured to activate the recombinase and/or the polymerase. Preferably, the metal cofactor composition comprises magnesium ions (e.g. magnesium acetate, magnesium chloride). The metal cofactor composition may be provided separately from the inorganic polyphosphate and/or the polyphosphate kinase (e.g. separately from the amplification composition as described herein). For example, the metal cofactor composition may be in a different container to the inorganic polyphosphate and/or the polyphosphate kinase (e.g. a different container to the amplification composition as described herein).

The kit may further comprise a solid support, preferably a flow cell. Preferably lawn primers (P5 and P7) are immobilised on the flow cell as described in detail above.

In a further embodiment, examples of the present disclosure are directed to use of an amplification composition as described herein, or a kit as described herein, in amplifying a nucleic acid template, or in sequencing a nucleic acid sequence.

In a further embodiment, examples of the present disclosure are directed to a method of amplifying a nucleic acid template, wherein the method comprises recycling ADP to ATP using inorganic polyphosphate and a polyphosphate kinase.

The method of amplifying a nucleic acid template may comprise adding an amplification composition as described herein. The compositions may be added to a sample containing a nucleic acid template to be amplified. In particular, by “adding” may mean that the compositions are added to a flow cell before, after or at the same time as a sample containing the nucleic acid template. The nucleic acid template may contain the adaptor sequences (comprising at least one of P5, P5′, P7 and P7′) as described above.

The method of amplifying a nucleic acid template may comprise adding a first polyphosphate composition and a second polyphosphate composition, wherein in the first polyphosphate composition, an amount of the first inorganic polyphosphate is higher relative to an amount of the second inorganic polyphosphate, and wherein in the second polyphosphate composition, an amount of the second inorganic polyphosphate is higher relative to an amount of the first inorganic polyphosphate.

The first polyphosphate composition may comprise a first inorganic polyphosphate as defined herein and a second inorganic polyphosphate as defined herein, wherein a ratio of the first inorganic polyphosphate to the second inorganic polyphosphate is about 99:1 to about 50:50, preferably about 98:2 to about 55:45, more preferably about 95:5 to about 60:40, even more preferably about 90:10 to about 65:35, yet even more preferably about 85:15 to about 70:30, most preferably about 80:20 to about 75:25.

The second polyphosphate composition may comprise a first inorganic polyphosphate as defined herein and a second inorganic polyphosphate as defined herein, wherein a ratio of the first inorganic polyphosphate to the second inorganic polyphosphate is about 50:50 to about 1:99, preferably about 45:55 to about 2:98, more preferably about 40:60 to about 5:95, even more preferably about 35:65 to about 10:90, yet even more preferably about 30:70 to about 15:85, most preferably about 25:75 to about 20:80.

Preferably, the first polyphosphate composition is added before the second polyphosphate composition.

Amplification may be conducted by exclusion amplification. Amplification may be conducted by bridge amplification.

Amplification may be conducted at a temperature of about 50° C. to about 75° C., about 55° C. to about 70° C., or about 60° C. to about 65° C. Preferably, amplification is conducted isothermally.

In a further embodiment, examples of the present disclosure are directed to a method of sequencing a nucleic acid sequence, wherein the method comprises amplifying a nucleic acid template as described herein; and sequencing the amplified nucleic acid template.

The step of sequencing the amplified nucleic acid template may comprise performing a single read. In other embodiments, the step of sequencing the amplified nucleic acid template comprises performing a paired-end read.

The step of sequencing the amplified nucleic acid template may comprise conducting a first sequencing read and a second sequencing read.

The step of sequencing the amplified nucleic acid template may be conducted using a sequencing-by-synthesis technique or a sequencing-by-ligation technique.

The method of sequencing a nucleic acid sequence may be conducted isothermally.

One or more steps in the method of sequencing a nucleic acid sequence are conducted at a temperature of about 50° C. to about 75° C., about 55° C. to about 70° C., or about 60° C. to about 65° C. Preferably, all steps in the method of sequencing a nucleic acid sequence are conducted at a temperature of about 50° C. to about 75° C., about 55° C. to about 70° C., or about 60° C. to about 65° C.

Where bridge amplification is used, the step of sequencing the amplified nucleic acid template may comprise a first linearisation step. The first linearisation step may be conducted after (e.g. immediately after) the step of amplifying a nucleic acid template.

The step of sequencing the amplified nucleic acid template may comprise a step of adding an exonuclease. The step of adding an exonuclease may be conducted after the step of amplifying a nucleic acid template. For example, the step of adding an exonuclease may be conducted after (e.g. immediately after) the first linearisation step.

Preferably, the exonuclease is a thermophilic exonuclease. More preferably, the exonuclease is derived from a thermophilic organism, such as Pyrococcus furious.

Preferably, the exonuclease has an optimum working temperature of about 50° C. to about 75° C., about 55° C. to about 70° C., or about 60° C. to about 65° C.

The step of sequencing the amplified nucleic acid template may comprise a first step of dehybridising a complementary strand bound to the nucleic acid template with a dehybridisation agent. The dehybridisation agent may be configured to cause the complementary strand to detach from the nucleic acid template and thereby allow the complementary strand to be washed away. The first step of dehybridising a complementary strand may be conducted after the step of amplifying a nucleic acid template. For example, the first step of dehybridising a complementary strand may be conducted after (e.g. immediately after) the step of adding an exonuclease.

The step of sequencing the amplified nucleic acid template may comprise a first step of hybridising a sequencing primer onto the nucleic acid template. The first step of hybridising a sequencing primer may be conducted after the step of amplifying a nucleic acid template. For example, the first step of hybridising a sequencing primer may be conducted after (e.g. immediately after) the first step of dehybridising a complementary strand.

The step of sequencing the amplified nucleic acid template may comprise a first step of performing sequencing-by-synthesis. The first step of performing sequencing-by-synthesis may be conducted after the step of amplifying a nucleic acid template. For example, the first step of performing sequencing-by-synthesis may be conducted after (e.g. immediately after) the first step of hybridising a sequencing primer.

Where a second sequencing read (e.g. for a paired-end read) is conducted, the step of sequencing the amplified nucleic acid may further comprise a step of removing a blocking group from a hydroxyl group of a primer (e.g. a P5 or a P7 primer). For example, the step of removing a blocking group may involve removal of a phosphate group using a blocking group phosphatase. The step of removing a blocking group may be conducted after the step of amplifying a nucleic acid template. For example, the step of removing a blocking group may be conducted after (e.g. immediately after) the first step of performing sequencing-by-synthesis.

Preferably, the blocking group phosphatase is a thermophilic phosphatase. More preferably, the blocking group phosphatase is derived from a thermophilic organism, such as Pyrococcus furious.

Preferably, the phosphatase has an optimum working temperature of about 50° C. to about 75° C., about 55° C. to about 70° C., or about 60° C. to about 65° C.

Where a second sequencing read (e.g. for a paired-end read) is conducted, the step of sequencing the amplified nucleic acid may further comprise a step of generating a complementary version of the amplified nucleic acid template. The step of generating a complementary version of the amplified nucleic acid template may involve using amplification methods as described herein, for example using inorganic polyphosphate and polyphosphate kinase. The step of generating a complementary version of the amplified nucleic acid template may be conducted after the step of amplifying a nucleic acid template. For example, the step of generating a complementary version of the amplified nucleic acid template may be conducted after (e.g. immediately after) the step of removing a blocking group.

Where a second sequencing read (e.g. for a paired-end read) is conducted, the step of sequencing the amplified nucleic acid template may comprise a second linearisation step. The second linearisation step may involve the use of an oxoguanine glycosylase (Ogg). The second linearisation step may be conducted after (e.g. immediately after) the step of generating a complementary version of the amplified nucleic acid template.

Preferably, the oxoguanine glycosylase is a thermophilic oxoguanine glycosylase. More preferably, the oxoguanine glycosylase is derived from a thermophilic organism, such as Methanococcus jannaschii.

Where a second sequencing read (e.g. for a paired-end read) is conducted, the step of sequencing the amplified nucleic acid template may comprise a second step of dehybridising a complementary strand bound to the (complementary version of the) nucleic acid template with a dehybridisation agent. The dehybridisation agent may be configured to cause the complementary strand to detach from the (complementary version of the) nucleic acid template and thereby allow the complementary strand to be washed away. The second step of dehybridising a complementary strand may be conducted after the step of amplifying a nucleic acid template. For example, the second step of dehybridising a complementary strand may be conducted after (e.g. immediately after) the second linearisation step.

Where a second sequencing read (e.g. for a paired-end read) is conducted, the step of sequencing the amplified nucleic acid template may comprise a second step of hybridising a sequencing primer onto the (complementary version of the) nucleic acid template. The second step of hybridising a sequencing primer may be conducted after the step of amplifying a nucleic acid template. For example, the second step of hybridising a sequencing primer may be conducted after (e.g. immediately after) the second step of dehybridising a complementary strand.

Where a second sequencing read (e.g. for a paired-end read) is conducted, the step of sequencing the amplified nucleic acid template may comprise a second step of performing sequencing-by-synthesis. The second step of performing sequencing-by-synthesis may be conducted after the step of amplifying a nucleic acid template. For example, the second step of performing sequencing-by-synthesis may be conducted after (e.g. immediately after) the second step of hybridising a sequencing primer.

The present disclosure will now be described by way of the following non-limiting examples.

### EXAMPLES

### Materials and Methods

### Tth PPK1 and Mru PPK2:

A screen was conducted for Tth PPK1 and Mm PPK2 to identify conditions for soluble protein expression. Tth PPK1 and Mm PPK2 plasmids (pET28a; which adds an in-frame N-terminal hexa-histidine purification tag) were transformed into Escherichia coli BL21 pLysS strain and plated onto Luria Bertani (LB) agar plates supplemented with 50 μg/ml kanamycin antibiotic for selection. A single colony was picked for each respective transformant and inoculated into LB broth supplemented 50 μg/ml kanamycin. The LB broth cultures were incubated at 37° C. at 225 rpm overnight. The overnight cultures were utilized as starter cultures at 1% inoculum into Terrific Broth (TB) medium supplemented with 50 μg/m1 kanamycin. The TB cultures were incubated 37° C. at 225 rpm until optical density (O.D.) 600 nm reached 0.6. A 5 ml aliquot of culture was removed for comparative analysis of the uninduced state for the recombinant gene of interest. The 37° C. temperature condition was induced with 1 mM final concentration of isopropyl thiogalactopyranoside (IPTG) and incubated for an additional three hours at 37° C. at 225 rpm. The 18° C. temperature condition cultures were incubated for 20 min in an ice water bath followed by induction with 1 mM final concentration of isopropyl thiogalactopyranoside (IPTG). These cultures were incubated for 16 hours at 18° C. rotating at 225 rpm.

The cultures were harvested at their respective times and pelleted by centrifugation. The supernatant was decanted, and the cells were frozen at −80° C. and then thawed to 22° C. The pellets were enzymatically lysed with lysonase supplemented in resuspension buffer (50 mM Tris pH 8.0, 500 mM NaCl, 25 mM imidazole, and 0.1 Triton-X100) incubating at 22° C. on a rocking platform. The lysed material was centrifuged at 18,000× g for 20 minutes at 4° C. The supernatant (soluble fraction) was removed and 4 μl was added to 20 μl of lysis buffer and 6 ul of 5X SDS-loading buffer. Analysis of the insoluble fraction was performed by taking a P20 pipette tip and aspirating ˜1 μl of the pellet and resuspending in 24 μl of lysis buffer and 6 ul of 5X SDS-loading buffer. The samples were heated to 95° C. for 5 minutes, cooled to 22° C., centrifuged, and loaded into an SDS-PAGE gel with a 4-12% gradient.

Mm PPK2 was taken forward for purification. The 18° C. overnight induction condition was purified immobilized metal affinity chromatography (IMAC). After lysis and clarification, the supernatant was applied to Ni-NTA purification column. The flow-through (FT) was collected and the column was washed with 10 column volumes (CV) of 50 mM Tris pH 8.0, 200 mM NaCl, and 25 mM imidazole. The wash was collected for subsequent analysis. Mm PPK2 was eluted from the IMAC column with 50 mM Tris pH 8.0, 200 mM NaCl, and 500 mM imidazole. Eluted fractions, FT, and wash fractions were assessed by SDS-PAGE (same procedure as described above). The fraction with the eluted protein was dialyzed against and 20 mM Tris pH 7.5, 300 mM NaCl, 0.5 mM DTT, 1 mM EDTA, and 50% glycerol and stored at −20° C.

### Reference Example 1

### Effect of Reducing PPi Using Pyrophosphatase (FIG. 5)

On board cluster generation (OBCG) was performed utilizing the NextSeq 2000 with a custom recipe to pull the ExAmp supplemented with 0.3 U PPiase per 100 μl clustering reagent or 1.2 U PPiase per 100 μl clustering reagent from a unique position within the sequencing cartridge. TruSeq Nano 450 (NA12878; source genomic DNA) supplemented with 1% PhiX v3 Control at a concentration of 300 pM was the seeded library. Two high output (HO) P3 flowcells and accompanying cartridges were utilized for each test condition. A single high output (HO) P3 flowcell was utilized as a control for comparison. A 2X151 sequencing run was executed for each of the flowcells. Primary metrics were pulled from sequence analysis viewer (SAV). The run was analyzed through the BaseSpace analysis workflow with DRAGEN Germline Alignment v3.7.5, downsample-bam, Firebrand R&D, which was automated with a wrapper in the AVATAR platform.

Overall, Reference Example 1 shows that improvements in various sequencing metrics can be obtained by reducing pyrophosphate levels.

### Example 2

### Amplification Using Polyphosphate Kinase and Inorganic Pyrophosphate (FIG. 6)

An in vitro reaction was conducted to screen for other energy supply systems for amplification reactions, as shown in lanes 1 to 5 in FIG. 6. A 990 base pair template was utilized (a PCR amplified template followed by purification). The purified template was quantified by A_{280 nm} and diluted to 10 nM input stock for the in vitro recombinase polymerase amplification (RPA). Forward and reverse primers to the template were added at a final concentration of 1 μM. ExAmp clustering reagent was utilized to amplify the template in solution in a 20 μl reaction volume with template and primers. The reaction was incubated at 37° C. for 30 minutes. At 30 minutes 3 μl was removed and added to 27 ul termination buffer (NEB 6X purple loading dye [1X2.5% Ficoll; 10 mM EDTA; 3.3 mM Tris pH 8; 0.08% EDTA; visualization dyes] and 0.8 U of Proteinase K) and incubated for 15 min at 55° C. followed by 80° C. for 10 min. Three microliters were loaded onto a 2.2% agarose TAE gel resolved by electrophoresis and visualized by ethidium bromide staining illuminated with ultraviolet light.

Lane 1 is a positive control showing a standard amplification mixture using creatine kinase and creatine phosphate, resulting in the production of a band at around 1000 bp. Lane 3 shows that PPK2 and PPi can be used as an energy system instead of creatine kinase and creatine phosphate systems. Negative control experiments show that the removal of various components as shown in lanes 2, 4 and 5 result in no amplification.

Overall, Example 2 shows that levels of pyrophosphate can be reduced using polyphosphate kinase.

### ADDITIONAL COMMENTS

While various illustrative examples are described above, it will be apparent to one skilled in the art that various changes and modifications may be made therein without departing from the disclosure. The appended claims are intended to cover all such changes and modifications that fall within the true spirit and scope of the embodiments described herein.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein

SEQUENCE LISTING
SEQ ID NO. 1:
Thermus thermophilus (Tth) Polyphosphate
Kinase 1 (PPK1):
HLLPEASWLQFNRRVLLQTERPDFPLLERLRFLGIWNRNL
DEFFAARIAKPFLKSRRGPDHLALLQEALDQAKLARARYQ
NLLQEAFPRLRVLDPGELDDLDWLYFRVFLAEEVAPKTDL
IPWEAAQDLSHSALYFASERYLVRLPQDLPRLVEVPGREG
TYVRLGALMRWRSDLLLPEEAPLYEFRVLRLLESERVRAD
WNELAESLEGRQEGTPTLLVVEEGFPEAWLDALRRALGLF
LEEVFALKPPLNLSLVDTLVAQGPPEWRFPPFRPERPRTF
LKNPLALLGKRDVLLYHPFEDYAAVERFAEAALAEEVEEV
WATLYRTGEENPLAEALIAAARKGKRVHVLLEGRARFDEL
LNLRWYLRLVRAGVEVLPLPERKVHAKAFLILTREGGYAH
LGTGNYNPINGHHYTDFSLFTARKEVVAEVRAFFQAMAEE
KTPRLGLLRTGEGIRRLLLEAVLHEAHPKGRLILKFNHLT
DPELLEALVYAASRGARVDLLVRSTLTRLHPAIRAKSLVG
RFLEHARAAAFRAGGEWRVYLTSADAMPRNFQNRFELLFP
VLDKEAKKKVLKVLKRQVRDDRNSFLLTPEGEKRLWGGRH
DAQRL
SEQ ID NO. 2:
Meiothermus ruber (Mru) Polyphosphate
Kinase 2 (PPK2):
KKYRVQPDGRFELKRFDPDDTSAFEGGKQAALEALAVLNR
RLEKLQELLYAEGQHKVLVVLQAMDAGGKDGTIRVVFDGV
NPSGVRVASFGVPTEQELARDYLWRVHQQVPRKGELVIFN
RSHYEDVLVVRVKNLVPQQVWQKRYRHIREFERMLADEGT
TILKFFLHISKDEQRQRLQERLDNPEKRWKFRMGDLEDRR
LWDRYQEAYEAAIRETSTEYAPWYVIPANKNWYRNWLVSH
ILVETLEGLAMQYPQPETASEKIVIE
SEQ ID NO. 3:
Escherichia coli Polyphosphate
Kinase 1 (PPK1):
MGQEKLYIEKELSWLSFNERVLQEAADKSNPLIERMRFLG
IYSNNLDEFYKVRFAELKRRIIISEEQGSNSHSRHLLGKI
QSRVLKADQEFDGLYNELLLEMARNQIFLINERQLSVNQQ
NWLRHYFKQYLRQHITPILINPDTDLVQFLKDDYTYLAVE
IIRGDTIRYALLEIPSDKVPRFVNLPPEAPRRRKPMILLD
NILRYCLDDIFKGFFDYDALNAYSMKMTRDAEYDLVHEME
ASLMELMSSSLKQRLTAEPVRFVYQRDMPNALVEVLREKL
TISRYDSIVPGGRYHNFKDFINFPNVGKANLVNKPLPRLR
HIWFDKAQFRNGFDAIRERDVLLYYPYHTFEHVLELLRQA
SFDPSVLAIKINIYRVAKDSRIIDSMIHAAHNGKKVTVVV
ELQARFDEEANIHWAKRLTEAGVHVIFSAPGLKIHAKLFL
ISRKENGEVVRYAHIGTGNFNEKTARLYTDYSLLTADARI
TNEVRRVFNFIENPYRPVTFDYLMVSPQNSRRLLYEMVDR
EIANAQQGLPSGITLKLNNLVDKGLVDRLYAASSSGVPVN
LLVRGMCSLIPNLEGISDNIRAISIVDRYLEHDRVYIFEN
GGDKKVYLSSADWMTRNIDYRIEVATPLLDPRLKQRVLDI
IDILFSDTVKARYIDKELSNRYVPRGNRRKVRAQLAIYDY
IKSLEQPE
SEQ ID NO. 4:
P5 sequence:
AATGATACGGCGACCACCGAGATCTACAC
SEQ ID NO. 5:
P7 sequence:
CAAGCAGAAGACGGCATACGAGAT
SEQ ID NO. 6:
P5′ sequence (complementary to P5):
GTGTAGATCTCGGTGGTCGCCGTATCATT
SEQ ID NO. 7:
P7′ sequence (complementary to P7):
ATCTCGTATGCCGTCTTCTGCTTG:
SEQ ID NO: 8:
RB32 UvsX with His tag:
MGSSHHHHHHSSGLVPRGSHMSIADLKSRLIKASTSKMTA
ELTTSKFFNEKDVIRTKIPMLNIAISGAIDGGMQSGLTIF
AGPSKHFKSNMSLTMVAAYLNKYPDAVCLFYDSEFGITPA
YLRSMGVDPERVIHTPIQSVEQLKIDMVNQLEAIERGEKV
IVFIDSIGNMASKKETEDALNEKSVADMTRAKSLKSLFRI
VTPYFSIKNIPCVAVNHTIETIEMFSKTVMTGGTGVMYSA
DTVFIIGKRQIKDGSDLQGYQFVLNVEKSRTVKEKSKFFI
DVKFDGGIDPYSGLLDMALELGFVVKPKNGWYAREFLDEE
TGEMIREEKSWRAKDTNCTTFWGPLFKHQPFRDAIKRAYQ
LGAIDSNEIVEAEVDELINSKVEKFKSPESKSKSAADLET
DLEQLSDMEEFNEGGHHHHH
SEQ ID NO: 9
RB32 UvsX:
MSIADLKSRLIKASTSKMTAELTTSKFFNEKDVIRTKIPM
LNIAISGAIDGGMQSGLTIFAGPSKHFKSNMSLTMVAAYL
NKYPDAVCLFYDSEFGITPAYLRSMGVDPERVIHTPIQSV
EQLKIDMVNQLEAIERGEKVIVFIDSIGNMASKKETEDAL
NEKSVADMTRAKSLKSLFRIVTPYFSIKNIPCVAVNHTIE
TIEMFSKTVMTGGTGVMYSADTVFIIGKRQIKDGSDLQGY
QFVLNVEKSRTVKEKSKFFIDVKFDGGIDPYSGLLDMALE
LGFVVKPKNGWYAREFLDEETGEMIREEKSWRAKDTNCTT
FWGPLFKHQPFRDAIKRAYQLGAIDSNEIVEAEVDELINS
KVEKFKSPESKSKSAADLETDLEQLSDMEEFNE
SEQ ID NO: 10
Thermophilic UvsX HQ:
MSIADLKSRLIKASTSKMTAELTTSKFFNEKDVIRTKIPM
LNIAISGAIDGGMQSGLTIFAGPSKSFKSNMSLTMVAAYL
NKYPDAVCLFYDSEFGITPAYLRSMGVDPERVIHTPIQSV
EQLKIDMVNQLEAIERGEKVIVFIDSIGNMASKKETEDAL
NEKSVADMTRAKSLKSLFRIVTPYFSIKNIPCVAVNHTIE
TIEMFSKTVMTGGTGVMYSADTVFIIGKRQIKDGSDLQGY
QFVLNVEKSRTVKEKSKFFIDVKFDGGIDPYSGLLDMALE
LGFVVKPKNGWYAREFLDEETGEMIREEKSWRAKDINCTT
FWGPLFKHQPFRDAIKRAYQLGAIDSNEIVEAEVDELINS
KVEKFKSPESKSKSAADLETDLEQLSDMEEFNEHQHQH
SEQ ID NO: 11
Thermophilic UvsX His:
MSIADLKSRLIKASTSKMTAELTTSKFFNEKDVIRTKIPM
LNIAISGAIDGGMQSGLTIFAGPSKSFKSNMSLTMVAAYL
NKYPDAVCLFYDSEFGITPAYLRSMGVDPERVIHTPIQSV
EQLKIDMVNQLEAIERGEKVIVFIDSIGNMASKKETEDAL
NEKSVADMTRAKSLKSLFRIVTPYFSIKNIPCVAVNHTIE
TIEMFSKTVMTGGTGVMYSADIVFIIGKRQIKDGSDLQGY
QFVLNVEKSRTVKEKSKFFIDVKFDGGIDPYSGLLDMALE
LGFVVKPKNGWYAREFLDEETGEMIREEKSWRAKDINCTT
FWGPLFKHQPFRDAIKRAYQLGAIDSNEIVEAEVDELINS
KVEKFKSPESKSKSAADLETDLEQLSDMEEFNEGGHHHHH

1. An amplification composition comprising an inorganic polyphosphate and a polyphosphate kinase.

2. The amplification composition according to claim 1, wherein the amplification composition comprises at least one selected from the group consisting of:
a recombinase, a single-stranded nucleotide binding protein, a polymerase and NTPs.

3. The amplification composition according to claim 1, wherein the amplification composition comprises a recombinase.

4. The amplification composition according to claim 1, wherein the amplification composition comprises a recombinase, a single-stranded nucleotide binding protein, a polymerase and NTPs.

5. The amplification composition according to claim 1, wherein the amplification composition comprises the inorganic polyphosphate at a concentration of about 0.01 μM to about 1000 μM.

6. The amplification composition according to claim 1, wherein the amplification composition comprises the polyphosphate kinase at a concentration of about 0.01 μM to about 1000 μM.

7. The amplification composition according to claim 1, wherein the inorganic polyphosphate comprises a first inorganic polyphosphate with less than 50 phosphate residues.

8. The amplification composition according to claim 1, wherein the inorganic polyphosphate comprises a second inorganic polyphosphate with more than 100 phosphate residues.

9. The amplification composition according to claim 8, wherein a ratio of the first inorganic polyphosphate to the second inorganic polyphosphate is about 90:10 to about 10:90.

10. The amplification composition according to claim 1, wherein the polyphosphate kinase is a thermophilic polyphosphate kinase.

11. The amplification composition according to claim 1, wherein the polyphosphate kinase has an optimum working temperature of about 50° C. to about 75° C.

12. The amplification composition according to claim 1, wherein the polyphosphate kinase is selected from the group consisting of: a polyphosphate kinase of the PPK1 family, a polyphosphate kinase of the PPK2 family, and a polyphosphate kinase of the PPK3 family.

13. The amplification composition according to claim 1, wherein the polyphosphate kinase comprises an amino acid sequence as defined in SEQ ID NO: 1 to 3, or a functional variant or functional fragment thereof.

14. The amplification composition according to claim 1, wherein the composition does not comprise PEG.

15. The amplification composition according to claim 1, wherein the amplification composition comprises a buffer.

16. The amplification composition according to claim 15, wherein the amplification composition is buffered to a pH of about 6.0 to about 9.0.

17. The amplification composition according to claim 1, wherein the composition is a clustering composition or a sequencing-by-synthesis amplification composition or a resynthesis composition.

18. A kit comprising an inorganic polyphosphate and a polyphosphate kinase.

19. The kit according to claim 18, wherein the kit further comprises a metal cofactor composition, wherein the metal cofactor composition comprises magnesium ions.

20. Use of an amplification composition according to claim 1, in amplifying a nucleic acid template, or in sequencing a nucleic acid sequence.

21. A method of amplifying a nucleic acid template, wherein the method comprises recycling ADP to ATP using inorganic polyphosphate and a polyphosphate kinase.

22. (canceled)

23. (canceled)

24. (canceled)

25. (canceled)

26. A method of sequencing a nucleic acid sequence, wherein the method comprises:
amplifying a nucleic acid template using the method of claim 21; and
sequencing the amplified nucleic acid template.

27. (canceled)

28. (canceled)

29. (canceled)

30. (canceled)


SEQUENCE LISTING (12 sequences)

SEQ ID NO: 1 - protein, 605 aa, synthetic construct
  source 1..605: note = polyphosphate kinase
HLLPEASWLQFNRRVLLQTERPDFPLLERLRFLGIWNRNLDEFFAARIAKPFLKSRRGPDHLALLQEALDQAKLARARYQNLLQEAFPRLRVLDPGELDDLDWLYFRVFLAEEVAPKTDLIPWEAAQDLSHSALYFASERYLVRLPQDLPRLVEVPGREGTYVRLGALMRWRSDLLLPEEAPLYEFRVLRLLESERVRADWNELAESLEGRQEGTPTLLVVEEGFPEAWLDALRRALGLFLEEVFALKPPLNLSLVDTLVAQGPPEWRFPPFRPERPRTFLKNPLALLGKRDVLLYHPFEDYAAVERFAEAALAEEVEEVWATLYRTGEENPLAEALIAAARKGKRVHVLLEGRARFDELLNLRWYLRLVRAGVEVLPLPERKVHAKAFLILTREGGYAHLGTGNYNPTNGHHYTDFSLFTARKEVVAEVRAFFQAMAEEKTPRLGLLRTGEGIRRLLLEAVLHEAHPKGRLILKFNHLTDPELLEALVYAASRGARVDLLVRSTLTRLHPAIRAKSLVGRFLEHARAAAFRAGGEWRVYLTSADAMPRNFQNRFELLFPVLDKEAKKKVLKVLKRQVRDDRNSFLLTPEGEKRLWGGRHDAQRL

SEQ ID NO: 2 - protein, 266 aa, synthetic construct
  source 1..266: note = polyphosphate kinase
KKYRVQPDGRFELKRFDPDDTSAFEGGKQAALEALAVLNRRLEKLQELLYAEGQHKVLVVLQAMDAGGKDGTIRVVFDGVNPSGVRVASFGVPTEQELARDYLWRVHQQVPRKGELVIFNRSHYEDVLVVRVKNLVPQQVWQKRYRHIREFERMLADEGTTILKFFLHISKDEQRQRLQERLDNPEKRWKFRMGDLEDRRLWDRYQEAYEAAIRETSTEYAPWYVIPANKNWYRNWLVSHILVETLEGLAMQYPQPETASEKIVIE

SEQ ID NO: 3 - protein, 688 aa, synthetic construct
  source 1..688: note = polyphosphate kinase
MGQEKLYIEKELSWLSFNERVLQEAADKSNPLIERMRFLGIYSNNLDEFYKVRFAELKRRIIISEEQGSNSHSRHLLGKIQSRVLKADQEFDGLYNELLLEMARNQIFLINERQLSVNQQNWLRHYFKQYLRQHITPILINPDTDLVQFLKDDYTYLAVEIIRGDTIRYALLEIPSDKVPRFVNLPPEAPRRRKPMILLDNILRYCLDDIFKGFFDYDALNAYSMKMTRDAEYDLVHEMEASLMELMSSSLKQRLTAEPVRFVYQRDMPNALVEVLREKLTISRYDSIVPGGRYHNFKDFINFPNVGKANLVNKPLPRLRHIWFDKAQFRNGFDAIRERDVLLYYPYHTFEHVLELLRQASFDPSVLAIKINIYRVAKDSRIIDSMIHAAHNGKKVTVVVELQARFDEEANIHWAKRLTEAGVHVIFSAPGLKIHAKLFLISRKENGEVVRYAHIGTGNFNEKTARLYTDYSLLTADARITNEVRRVFNFIENPYRPVTFDYLMVSPQNSRRLLYEMVDREIANAQQGLPSGITLKLNNLVDKGLVDRLYAASSSGVPVNLLVRGMCSLIPNLEGISDNIRAISIVDRYLEHDRVYIFENGGDKKVYLSSADWMTRNIDYRIEVATPLLDPRLKQRVLDIIDILFSDTVKARYIDKELSNRYVPRGNRRKVRAQLAIYDYIKSLEQPE

SEQ ID NO: 4 - DNA, 29 bp, synthetic construct
  source 1..29: mol_type = other DNA; note = P5
aatgatacggcgaccaccgagatctacac

SEQ ID NO: 5 - DNA, 24 bp, synthetic construct
  source 1..24: mol_type = other DNA; note = P7
caagcagaagacggcatacgagat

SEQ ID NO: 6 - DNA, 29 bp, synthetic construct
  source 1..29: mol_type = other DNA; note = P5 prime
gtgtagatctcggtggtcgccgtatcatt

SEQ ID NO: 7 - DNA, 24 bp, synthetic construct
  source 1..24: mol_type = other DNA; note = P7 prime
atctcgtatgccgtcttctgcttg

SEQ ID NO: 8 - protein, 420 aa, synthetic construct
  source 1..420: note = RB32 UvsX with His tag
MGSSHHHHHHSSGLVPRGSHMSIADLKSRLIKASTSKMTAELTTSKFFNEKDVIRTKIPMLNIAISGAIDGGMQSGLTIFAGPSKHFKSNMSLTMVAAYLNKYPDAVCLFYDSEFGITPAYLRSMGVDPERVIHTPIQSVEQLKIDMVNQLEAIERGEKVIVFIDSIGNMASKKETEDALNEKSVADMTRAKSLKSLFRIVTPYFSIKNIPCVAVNHTIETIEMFSKTVMTGGTGVMYSADTVFIIGKRQIKDGSDLQGYQFVLNVEKSRTVKEKSKFFIDVKFDGGIDPYSGLLDMALELGFVVKPKNGWYAREFLDEETGEMIREEKSWRAKDTNCTTFWGPLFKHQPFRDAIKRAYQLGAIDSNEIVEAEVDELINSKVEKFKSPESKSKSAADLETDLEQLSDMEEFNEGGHHHHH

SEQ ID NO: 9 - protein, 393 aa, synthetic construct
  source 1..393: note = RB32 UvsX
MSIADLKSRLIKASTSKMTAELTTSKFFNEKDVIRTKIPMLNIAISGAIDGGMQSGLTIFAGPSKHFKSNMSLTMVAAYLNKYPDAVCLFYDSEFGITPAYLRSMGVDPERVIHTPIQSVEQLKIDMVNQLEAIERGEKVIVFIDSIGNMASKKETEDALNEKSVADMTRAKSLKSLFRIVTPYFSIKNIPCVAVNHTIETIEMFSKTVMTGGTGVMYSADTVFIIGKRQIKDGSDLQGYQFVLNVEKSRTVKEKSKFFIDVKFDGGIDPYSGLLDMALELGFVVKPKNGWYAREFLDEETGEMIREEKSWRAKDTNCTTFWGPLFKHQPFRDAIKRAYQLGAIDSNEIVEAEVDELINSKVEKFKSPESKSKSAADLETDLEQLSDMEEFNE

SEQ ID NO: 10 - protein, 396 aa, synthetic construct
  source 1..396: note = Thermophilic UvsX HQ
MSIADLKSRLIKASTSKMTAELTTSKFFNEKDVIRTKIPMLNIAISGAIDGGMQSGLTIFAGPSKSFKSNMSLTMVAAYLNKYPDAVCLFYDSEFGITPAYLRSMGVDPERVIHTPIQSVEQLKIDMVNQLEAIERGEKVIVFIDSIGNMASKKETEDALNEKSVADMTRAKSLKSLFRIVTPYFSIKNIPCVAVNHTTIEMFSKTVMTGGTGVMYSADTVFIIGKRQIKDGSDLQGYQFVLNVEKSRTVKEKSKFFIDVKFDGGIDPYSGLLDMALELGFVVKPKNGWYAREFLDEETGEMIREEKSWRAKDINCTTFWGPLFKHQPFRDAIKRAYQLGAIDSNEIVEAEVDELINSKVEKFKSPESKSKSAADLETDLEQLSDMEEFNEHQHQH

SEQ ID NO: 11 - protein, 400 aa, synthetic construct
  source 1..400: note = Thermophilic UvsX His
MSIADLKSRLIKASTSKMTAELTTSKFFNEKDVIRTKIPMLNIAISGAIDGGMQSGLTIFAGPSKSFKSNMSLTMVAAYLNKYPDAVCLFYDSEFGITPAYLRSMGVDPERVIHTPIQSVEQLKIDMVNQLEAIERGEKVIVFIDSIGNMASKKETEDALNEKSVADMTRAKSLKSLFRIVTPYFSIKNIPCVAVNHTIETIEMFSKTVMTGGTGVMYSADIVFIIGKRQIKDGSDLQGYQFVLNVEKSRTVKEKSKFFIDVKFDGGIDPYSGLLDMALELGFVVKPKNGWYAREFLDEETGEMIREEKSWRAKDINCTTFWGPLFKHQPFRDAIKRAYQLGAIDSNEIVEAEVDELINSKVEKFKSPESKSKSAADLETDLEQLSDMEEFNEGGHHHHH

SEQ ID NO: 12 - protein, 400 aa, synthetic construct
  source 1..400: note = Consensus/UvsX 6His
MSIADLKSRLIKASTSKMTAELTTSKFFNEKDVIRTKIPMLNIAISGAIDGGMQSGLTIFAGPSKSFKSNMSLTMVAAYLNKYPDAVCLFYDSEFGITPAYLRSMGVDPERVIHTPIQSVEQLKIDMVNQLEAIERGEKVIVFIDSIGNMASKKETEDALNEKSVADMTRAKSLKSLFRIVTPYFSIKNIPCVAVNHTIETIEMFSKTVMTGGTGVMYSADTVFIIGKRQIKDGSDLQGYQFVLNVEKSRTVKEKSKFFIDVKFDGGIDPYSGLLDMALELGFVVKPKNGWYAREFLDEETGEMIREEKSWRAKDINCTTFWGPLFKHQPFRDAIKRAYQLGAIDSNEIVEAEVDELINSKVEKFKSPESKSKSAADLETDLEQLSDMEEFNEGGHHHHH